SEQUENCE LISTING (63 sequences)

SEQ ID NO: 1 - DNA, 6441 bp, Homo sapiens
  source 1..6441: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaa

SEQ ID NO: 2 - DNA, 6429 bp, Homo sapiens
  source 1..6429: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaa

SEQ ID NO: 3 - DNA, 4257 bp, Homo sapiens
  source 1..4257: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaa

SEQ ID NO: 4 - DNA, 4553 bp, Homo sapiens
  source 1..4553: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatgtgagtaatagaaggaattttatgtagagcacagtcagacgaactcgaaaaaatagaaaagcatggccgatcttccaaagacaaggaaaatgccaagtc

SEQ ID NO: 5 - DNA, 4467 bp, Homo sapiens
  source 1..4467: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagtgacactcatcccccctaccctcgccatgattcttaccttcttcccgatgacccctgaaaagaggagaaaaaggtgagaagggccaaagaaattttacacaatgggcagaacttctgtcggagaagatatcaagggtgtagagatcttccagaaaagggaaa

SEQ ID NO: 6 - DNA, 5838 bp, Homo sapiens
  source 1..5838: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaatgctaaaagcaccagcatactctatggatggagcaagaaatcaaatcttgtccctataaaagggccagaaagaaacatcatgccattggccaaagagcaggaagtgaagcaaaacgcctgaataccgaatgaccatttactaaatcattaaaagtttattatgtgattctagtcaataaacatatgctcagtgattcatttgttttctgaatatttaacattttctctatcaaacttttcatctaaatttctcataggcattttgttgttgttataactacaaaggaagggaactattttggttgttatttgtttttcataatttccctgaaattgagcaatacataagacagaatattcagatggaccttaccgtctaaggtttgctttctagttaacaatttgaaaactaatttattctctttatgttccgtctgcccatattaattatatttgataacacagggcgttaaggaaatagtggggaaaatgaggcatcgtgtgtctcattccttggtaggagtgtgctctccatatggctgaggggtagtgtggaatagtggtcaagggggtagagcctggaaccagactgcctggctcatttcctagctctgccactttatggatatgtgaccttgaacagccacttctgctgtaaaatgtggaccgtagtggtacctgtttcactggattttggcagtgattacaaggaagaataaactaaagccctttattatgcaccaaataatgttctctgaatacatacttgctacaacataataataaatatgcacatggtctcaataagagtcttttattcctttgtggttgtcattaaccttattatctgcatcacgtcttttatttgtgatgattaatatctctcattgaggtaacgaagtacaaagtgagatgctgatgcctgtattttgtagacacatacttgatatccaaatatttttggcaatatcatgaaacatttagatatggtactcacaatgtattggttttcttatattttaatttttattgtaaactttgatgcttctaactgaattctaagtgctgaaaacataatatgaaaattatagataggatacatatctaaaaccagatttttagagggaattctgaagggaaaaatatagctcagcagacttgtatgcaaccagataggtgaaacattgttttctcctttccaagatatttgaaatagcaaaggtccaaattatatcttgcaaatgtttttcatgccaaaattggctagactggcaaaaagatatgttcaaagaataaaagattgtaatgttattttaatttcaaggagtttaaacatctgtgcatattttctttttgctttaaagagtggagacttaggatagttggtttaaattagaaaggcattttgttttaagagcctgctgatactgcagcattatagtgcatgttactgtgatatttttaaacaaactgtgaaatttagaaatgcaacctgctagaataaaaactttaataaaatatatacctaatgcca

SEQ ID NO: 7 - DNA, 5826 bp, Homo sapiens
  source 1..5826: mol_type = genomic DNA
gcagccgcagccgcagcgacggcagccacgggagccgccgcgcattatgcaaagcggcggcagatgcgagcggggccagccgggcgcgcgtcggcctcccctcccagcggctccccccgccgccgcctgactctcccgggagactccctaggcccggacctggggccgaggagggccgggatggcctgagtgcccgcggcgcggcggcgcagcagcgggattgcaccatggggaaccaggatgggaagctgaagaggagcgcaggtgatgctttgcacgaaggcggcggtggcgccgaggatgcgctggggcccagggatgtggaagccacaaagaaggggagcgggggcaagaaggcgctaggcaagcacggcaaggggggagggggcggcggcggcggcggggagtcgggcaagaagaagagcaagtccgactccagagcctcggtgttttccaacctgcggatcaggaagaatctgtccaaggggaaaggcgccggcggctcccgcgaagatgtactggattcccaggccctgcagaccggggagctggacagcgctcactccctgctcaccaagactccagacctcagcctctcggcggacgaggccggcctgtcggataccgagtgtgcggacccttttgaggtgaccggtccagggggtcctgggcctgccgaggctagggtcgggggccggccgatcgccgaggatgtggaaactgcagcaggggcgcaggatggacaaaggaccagctcgggctcggacacggacatctatagcttccattcggctacggagcaagaggatttgctttcagacatccagcaggcgatccgcctgcagcagcagcagcagcagcagctccagctccagctccagcaacagcagcagcagcagcagctccagggcgccgaggagcctgcagcgccccccactgccgtctcccctcagcccggggccttcctgggcctggaccggttcctgctggggccgagcggcggggctggggaggccccgggcagtccggacaccgagcaggcgctgtccgcgctctccgacctgcccgagagcctggccgccgagccccgggagccccagcaaccgccgtcccccggcggcctcccggtctccgaggcgcccagtctcccggcagcgcaacccgcggccaaagactcgccctcctccacggctttcccatttcccgaggccgggccgggggaggaagcggccggagcccccgtgcgaggggctggggacacggatgaggagggtgaggaggatgcttttgaggatgcgccccggggctctccgggggaggagtgggccccggaggtgggagaggacgccccgcagaggctgggggaagagccggaggaggaggcgcaaggacctgacgcccccgcggccgcttccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggccgacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaatgctaaaagcaccagcatactctatggatggagcaagaaatcaaatcttgtccctataaaagggccagaaagaaacatcatgccattggccaaagagcaggaagtgaagcaaaacgcctgaataccgaatgaccatttactaaatcattaaaagtttattatgtgattctagtcaataaacatatgctcagtgattcatttgttttctgaatatttaacattttctctatcaaacttttcatctaaatttctcataggcattttgttgttgttataactacaaaggaagggaactattttggttgttatttgtttttcataatttccctgaaattgagcaatacataagacagaatattcagatggaccttaccgtctaaggtttgctttctagttaacaatttgaaaactaatttattctctttatgttccgtctgcccatattaattatatttgataacacagggcgttaaggaaatagtggggaaaatgaggcatcgtgtgtctcattccttggtaggagtgtgctctccatatggctgaggggtagtgtggaatagtggtcaagggggtagagcctggaaccagactgcctggctcatttcctagctctgccactttatggatatgtgaccttgaacagccacttctgctgtaaaatgtggaccgtagtggtacctgtttcactggattttggcagtgattacaaggaagaataaactaaagccctttattatgcaccaaataatgttctctgaatacatacttgctacaacataataataaatatgcacatggtctcaataagagtcttttattcctttgtggttgtcattaaccttattatctgcatcacgtcttttatttgtgatgattaatatctctcattgaggtaacgaagtacaaagtgagatgctgatgcctgtattttgtagacacatacttgatatccaaatatttttggcaatatcatgaaacatttagatatggtactcacaatgtattggttttcttatattttaatttttattgtaaactttgatgcttctaactgaattctaagtgctgaaaacataatatgaaaattatagataggatacatatctaaaaccagatttttagagggaattctgaagggaaaaatatagctcagcagacttgtatgcaaccagataggtgaaacattgttttctcctttccaagatatttgaaatagcaaaggtccaaattatatcttgcaaatgtttttcatgccaaaattggctagactggcaaaaagatatgttcaaagaataaaagattgtaatgttattttaatttcaaggagtttaaacatctgtgcatattttctttttgctttaaagagtggagacttaggatagttggtttaaattagaaaggcattttgttttaagagcctgctgatactgcagcattatagtgcatgttactgtgatatttttaaacaaactgtgaaatttagaaatgcaacctgctagaataaaaactttaataaaatatatacctaatgcca

SEQ ID NO: 8 - DNA, 4679 bp, Homo sapiens
  source 1..4679: mol_type = genomic DNA
ggagagatttaacatagctattttaaaatgtgagtctgataacaatctaggatccctgtgtgtctttttctgttgtctgttttcctattgatttttgtttattttttatttatgtctccatttttctattttttttttttattttatgctgtgcaaatgcttagtttgatctcatcttttcccagataggatttatgtttgcttttgccggggacctggggcactaatattccaagatcaccttgagcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaa

SEQ ID NO: 9 - DNA, 5137 bp, Homo sapiens
  source 1..5137: mol_type = genomic DNA
attagctctgtgcacttgggctttttgctcaggtagatgtatcggaaggttagtgagggtgggaattgaaggtatcagcctaaaggggtggtttaaagagtaggctacaaagtcagaagaccagagagggtgaatggtagaaaggaggggagccaagattctcaaggtctggaggctgagggggttcggataggatgcctacaggtattattgggtagataaattggtttcagttggtaagctgctggagggtgtggcctggtgagtggtctgctggctaagattgcgtgaaggtgagtgcttgcattggcttactctcactttctttcttcctctgggtgtgaatgatttgctaatcatggttcccattccagtgtgtaaccagtttgcaggtcattgtcagttttttttgtactggtgctttaaaaaacaaacaaactgttttcagtatttaaaaacacattaagctttctatgtaccaggctctgttttaagcactttacaattattaactgttttaatcttcattacaagcctggtgagatagtttttattttccttttgcagaatacactgaagcacaagaaaattacgttcattttcttccttgctttgaagttccattaccaacacaaaaccttcaactgcatttttgcatttctggctatctctgaccctaacttgccacaacacagatccacgcagcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaggatgatggagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctccgcccccacttcccggagcgggcataccccctcctccccctcttcccggagcgggcatacctcctccaccccctctacccggagcgggcataccccctccgcccccacttcccggagcgggcatacccccacctccccctctacccggagcgggcataccccctccgccccctctacccggagtgggcatacctcctccgccccctctacccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctacccggagcgggcataccccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaagagactccagtacttcacttatttgggaaaaaattgaagagccatccatagattgtcatgaatttgaggaattattttctaaaactgctgtaaaggagagaaagaaacctatctctgatactatctcaaagacgaaggctaaacaagttgtcaagttattaagcaacaaaagatcacaagcagttggaatactaatgtctagccttcatttagatatgaaagacatacaacatgctgttgtgaacttggataattctgtggttgacctggagacccttcaagctctctatgagaatagagcacagtcagacgaactcgaaaaaatagaaaagcatggccgatcttccaaagacaaggaaaatgccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagatacttgcaaaa

SEQ ID NO: 10 - DNA, 21 bp, Homo sapiens
  source 1..21: mol_type = genomic DNA
cagtcccaaggacgttgatac

SEQ ID NO: 11 - DNA, 21 bp, Homo sapiens
  source 1..21: mol_type = genomic DNA
acgccaagtctctcgacaaac

SEQ ID NO: 12 - DNA, 21 bp, Homo sapiens
  source 1..21: mol_type = genomic DNA
ggacagcgctcactctatagt

SEQ ID NO: 13 - DNA, 20 bp, Mus musculus
  source 1..20: mol_type = genomic DNA
ctaattgtggctgcccttgt

SEQ ID NO: 14 - DNA, 20 bp, Mus musculus
  source 1..20: mol_type = genomic DNA
aggtggcaatgtcaggattc

SEQ ID NO: 15 - DNA, 20 bp, Mus musculus
  source 1..20: mol_type = genomic DNA
cttgggtggagaggctattc

SEQ ID NO: 16 - DNA, 20 bp, Mus musculus
  source 1..20: mol_type = genomic DNA
aggtgagatgacaggagatc

SEQ ID NO: 17 - DNA, 19 bp, Mus musculus
  source 1..19: mol_type = genomic DNA
cctcccaattggtttccgc

SEQ ID NO: 18 - DNA, 20 bp, Mus musculus
  source 1..20: mol_type = genomic DNA
ccaagtctttgagccgcctg

SEQ ID NO: 19 - DNA, 20 bp, Mus musculus
  source 1..20: mol_type = genomic DNA
aaagaccagcatttccccgt

SEQ ID NO: 20 - DNA, 20 bp, Mus musculus
  source 1..20: mol_type = genomic DNA
gggaagccaaacaccctttc

SEQ ID NO: 21 - DNA, 20 bp, Mus musculus
  source 1..20: mol_type = genomic DNA
tgtgccagaccttggaaacc

SEQ ID NO: 22 - DNA, 23 bp, Mus musculus
  source 1..23: mol_type = genomic DNA
tgtctcccttcattgtttcctga

SEQ ID NO: 23 - DNA, 20 bp, Mus musculus
  source 1..20: mol_type = genomic DNA
ctgatgaatgtggaccccga

SEQ ID NO: 24 - DNA, 20 bp, Mus musculus
  source 1..20: mol_type = genomic DNA
cctctgcatgcctgataccc

SEQ ID NO: 25 - DNA, 20 bp, Mus musculus
  source 1..20: mol_type = genomic DNA
gaagccgaggaggtgtgtag

SEQ ID NO: 26 - DNA, 20 bp, Mus musculus
  source 1..20: mol_type = genomic DNA
atgctgcgcaagttgtttca

SEQ ID NO: 27 - DNA, 20 bp, Mus musculus
  source 1..20: mol_type = genomic DNA
ccttggctgggaggatcaat

SEQ ID NO: 28 - DNA, 20 bp, Mus musculus
  source 1..20: mol_type = genomic DNA
cttggtgtccgtcctgtgag

SEQ ID NO: 29 - DNA, 20 bp, Mus musculus
  source 1..20: mol_type = genomic DNA
gtctggaagccagtaacccg

SEQ ID NO: 30 - DNA, 20 bp, Mus musculus
  source 1..20: mol_type = genomic DNA
cggtcaccacgtcaatctct

SEQ ID NO: 31 - DNA, 20 bp, Mus musculus
  source 1..20: mol_type = genomic DNA
gcattatgcgaccctggtga

SEQ ID NO: 32 - DNA, 20 bp, Mus musculus
  source 1..20: mol_type = genomic DNA
caccgcattgggtacagaga

SEQ ID NO: 33 - DNA, 20 bp, Mus musculus
  source 1..20: mol_type = genomic DNA
agcaacctgctctcaatggt

SEQ ID NO: 34 - DNA, 20 bp, Mus musculus
  source 1..20: mol_type = genomic DNA
gtcctgtctcgtgagcttcc

SEQ ID NO: 35 - DNA, 20 bp, Mus musculus
  source 1..20: mol_type = genomic DNA
cagggggtccaaagtgctta

SEQ ID NO: 36 - DNA, 20 bp, Mus musculus
  source 1..20: mol_type = genomic DNA
agatgccattgcccccattc

SEQ ID NO: 37 - DNA, 18 bp, Mus musculus
  source 1..18: mol_type = genomic DNA
tggaaggcacagcgaccg

SEQ ID NO: 38 - DNA, 22 bp, Mus musculus
  source 1..22: mol_type = genomic DNA
agtcaggaggcatgtgatgatg

SEQ ID NO: 39 - DNA, 20 bp, Mus musculus
  source 1..20: mol_type = genomic DNA
gaggcggcgagaaagaaata

SEQ ID NO: 40 - DNA, 22 bp, Mus musculus
  source 1..22: mol_type = genomic DNA
cacactctccagtttctctgac

SEQ ID NO: 41 - DNA, 20 bp, Mus musculus
  source 1..20: mol_type = genomic DNA
caacagagaacctgctcttc

SEQ ID NO: 42 - DNA, 18 bp, Mus musculus
  source 1..18: mol_type = genomic DNA
gcagctgaggaggtacag

SEQ ID NO: 43 - DNA, 20 bp, Mus musculus
  source 1..20: mol_type = genomic DNA
ccttctacgacgatgccctc

SEQ ID NO: 44 - DNA, 22 bp, Mus musculus
  source 1..22: mol_type = genomic DNA
ggttcaaggtcatgctctgttt

SEQ ID NO: 45 - DNA, 21 bp, Mus musculus
  source 1..21: mol_type = genomic DNA
ctctgttctgctcttgcttct

SEQ ID NO: 46 - DNA, 21 bp, Mus musculus
  source 1..21: mol_type = genomic DNA
gctttgacctttcctcctaca

SEQ ID NO: 47 - DNA, 23 bp, Mus musculus
  source 1..23: mol_type = genomic DNA
tgaaagagctaggagtaggactg

SEQ ID NO: 48 - DNA, 21 bp, Mus musculus
  source 1..21: mol_type = genomic DNA
ctctcggttttgaccgtgatg

SEQ ID NO: 49 - DNA, 20 bp, Mus musculus
  source 1..20: mol_type = genomic DNA
acagtgaagaccaaggaagg

SEQ ID NO: 50 - DNA, 19 bp, Mus musculus
  source 1..19: mol_type = genomic DNA
ctggagggagcgtgtattt

SEQ ID NO: 51 - DNA, 20 bp, Mus musculus
  source 1..20: mol_type = genomic DNA
cgagcctgtacgacgaagtg

SEQ ID NO: 52 - DNA, 21 bp, Mus musculus
  source 1..21: mol_type = genomic DNA
aagctcaggtcgaacatgagg

SEQ ID NO: 53 - DNA, 21 bp, Mus musculus
  source 1..21: mol_type = genomic DNA
tggtgtcaagccggaagataa

SEQ ID NO: 54 - DNA, 19 bp, Mus musculus
  source 1..19: mol_type = genomic DNA
gctggtgcatcacccttct

SEQ ID NO: 55 - DNA, 20 bp, Mus musculus
  source 1..20: mol_type = genomic DNA
catggccttccgtgttccta

SEQ ID NO: 56 - DNA, 22 bp, Mus musculus
  source 1..22: mol_type = genomic DNA
cctgcttcaccaccttcttgat

SEQ ID NO: 57 - DNA, 21 bp, synthetic construct
  source 1..21: mol_type = other DNA
cctaaggttaagtcgccctcg

SEQ ID NO: 58 - DNA, 21 bp, Mus musculus
  source 1..21: mol_type = genomic DNA
cagtcccaaggacgttgatac

SEQ ID NO: 59 - DNA, 21 bp, Mus musculus
  source 1..21: mol_type = genomic DNA
acgccaagtctctcgacaaac

SEQ ID NO: 60 - DNA, 21 bp, Mus musculus
  source 1..21: mol_type = genomic DNA
ggacagcgctcactctatagt

SEQ ID NO: 61 - DNA, 21 bp, Mus musculus
  source 1..21: mol_type = genomic DNA
gacgcctccctcctacaaatt

SEQ ID NO: 62 - DNA, 21 bp, Mus musculus
  source 1..21: mol_type = genomic DNA
ccgtagccatcacagctaaac

SEQ ID NO: 63 - DNA, 21 bp, Mus musculus
  source 1..21: mol_type = genomic DNA
catcgctgagaacggctttac

(19) United States
(12) Patent Application Publication
(10) Pub. No.: US 2025/0122253 A1
(43) Pub. Date: Apr. 17, 2025

(54) REGULATING MICROTUBULE DYNAMICS AS A THERAPEUTIC TARGET FOR NERVE REPAIR

(71) Applicant: City University of Hong Kong, Hong Kong (CN)

(72) Inventors: Chi Him Eddie MA, Hong Kong (CN); Ngan Pan Bennett AU, Hong Kong (CN)

(21) Appl. No.: 18/437,404

(22) Filed: Feb. 9, 2024

Related U.S. Application Data
(60) Provisional application No. 63/590,594, filed on Oct. 16, 2023.

(51) Int. Cl. C07K 14/47; A61K 9/00; A61K 31/421; A61P 25/02

(52) U.S. Cl. CPC C07K 14/47; A61K 9/0053; A61K 9/0085; A61K 31/421; A61P 25/02

(57) ABSTRACT

Method of treating a nerve injury in a subject in need thereof, the method comprising: administering a therapeutically effective amount of a therapeutic agent to the subject, wherein the therapeutic agent is selected from the group consisting of metaxalone and a Formin-2 inhibitor, such as an antibody, an antibody fragment, an inhibitory nucleic acid molecule, or a small molecule, wherein the inhibitory nucleic acid molecule substantially silences Fmn2.

### CROSS-REFERENCE TO RELATED APPLICATIONS

The present application claims priority from U.S. Provisional Patent Application No. 63/590,594, filed on Oct. 16, 2023, which is hereby incorporated by reference in its entirety.

### REFERENCE TO SEQUENCE LISTING

The Sequence Listing identified as Sequence_Listing_P25599US01.xml; Size: 101 kilobytes; and Date of Creation: Jan. 9, 2024, filed herewith, is hereby incorporated by reference in its entirety.

### TECHNICAL FIELD

The present disclosure generally relates to the use of metaxalone and a Formin-2 inhibitor in the treatment of nerve injuries.

### BACKGROUND

Injury to the peripheral nervous system (PNS) induces either adaptive (growth and survival) or maladaptive (cell death, neuropathic pain, and failure of regeneration) responses determined precisely by expression levels of a subset of genes (intrinsic growth program). The first step for successful peripheral nerve regeneration is to induce axon regeneration from injured neurons. On the level of the cytoskeleton, this requires a precise balance between stable and dynamic microtubules, as is observed during neuronal development. Damage to the peripheral nerve is followed by a slow rate of axonal regrowth and associated with sub-optimal function recovery. For instance, the most common type of proximal nerve injury in humans such as brachial plexus injury and those that involve complete transection of peripheral nerve require long-distance axon regeneration to re-innervate their target muscles. The function recovery is virtually non-existent even after surgical repair due largely to the slow rate of axonal regrowth and limited target muscle reinnervation. One of the major challenges is therefore to identify gene(s) that are involved in this adaptive response.

DNA methylation is the most studied and common epigenetic modification in gene expression. Recent reports have demonstrated that the orchestration of global DNA methylation and demethylation serve as fundamental mechanisms to promote axon regeneration. Epigenetic regulators such as ten-eleven translocation methylcytosine dioxygenases (Tets) and ubiquitin-like containing PHD ring finger 1 (UHRF1) have been shown to be important for peripheral nerve regeneration. Tets are enzymes that regulate DNA demethylation and their expression is increased in dorsal root ganglion (DRGs) after sciatic nerve injury. TET3 was highly upregulated in DRG neurons at 1-3 days after sciatic nerve injury and promoted expression of multiple regeneration-associated genes (RAGs). UHRF1 enhanced regenerative capacity of DRG neurons through promoter methylation (gene silencing) of PTEN and REST. Nevertheless, the correlation between global DNA methylation and RAG expression after peripheral nerve injury (PNI) is weak and rather descriptive. Inconclusive results are reported regarding the importance of DNA methylation after peripheral axonal injury. Pharmacological perturbation of the PNI-induced change in DNA methylation status (either activation or inhibition of DNA methylation) resulted in a marked attenuation of axonal regrowth in DRG neurons. The administration of folate, which increased global DNA methylation in the spinal cord, enhanced axon regeneration of spinal neurons after spinal cord injury. However, how alternation of DNA methylation might functionally impact individual gene expression and orchestrate downstream effectors contributing to successful axon regeneration remains elusive.

Together with DNA methylation, histone modifications represent the classic and essential epigenetic mechanisms. Histone deacetylases (HDACs) are not only enzymes that catalyse deacetylation from histone in the nuclei but also from many cytoplasmic proteins such as α-tubulin. Post-translational modification of tubulin on microtubule dynamics, such as acetylation, which is one of the key events in regulating axon regeneration. Previous studies reported that inhibition of microtubule dynamics without disrupting the integrity of axonal microtubules results in reduction of axonal growth. Tubulin is the major building block of microtubules. Stable and dynamic microtubules can be distinguished by the extent of tubulin acetylation along the axon shaft. For instance, acetylated tubulins (stable) are mainly found in stable microtubules in the proximal segments of axons where active growth is limited. Highly deacetylated tubulins (dynamic) are located in the distal end of axons where active tubulin polymerization occurs at the growth cones. PNI induces microtubule remodeling to facilitate the formation of growth cones and promote axon regeneration. PNI induces tubulin deacetylation at the distal end of injured peripheral nerve where active axonal regrowth is taking place. The administration of scriptaid, a pan-HDAC inhibitor which blocks tubulin deacetylation in damaged axons, markedly stalled axonal regrowth after PNI.

The beneficial effects of low-dose ionizing radiation (LDIR) has been documented for decades. LDIR induces adaptive responses to enhance overall functional ability of organisms and their intrinsic growth capacity. The beneficial effects of LDIR such as X-ray on cell growth stimulation, average life span extension in rodent, neuroprotection in animal disease models as well as in human leukemia disease and Alzheimer's disease have been reported. Local low-dose X-ray irradiation of sciatic nerve improves nerve repair and function recovery in rats although the mechanism remains unclear. LDIR has been known to induce DNA methylation and transcriptional changes in different in vitro and in vivo models. These studies suggest that alternation in gene expression profiling following LDIR contributes to the beneficial adaptive response in living organisms. Given that there is still much to learn before LDIR becomes a routine, effective and safe medical treatment. By identifying key genes and small molecules that are associated with LDIR-induced protective effects could become an attractive and safe pharmacological treatment for PNIs.

There thus exists a need for improved methods for treating nerve injuries.

### SUMMARY

Herein it is shown that two commonly-used ionizing radiation sources, α-particle and X-ray, promoted neurite outgrowth in purified DRG neurons in cultures. LD whole-body X-ray irradiation enhanced the intrinsic growth capacity of injured neurons and induced robust axon regeneration in axotomized DRG neurons in ex vivo explant cultures, as well as accelerated in vivo axon regeneration and function recovery after sciatic nerve crush injury. In-depth genome-wide CpG methylation profiling and bioinformatic analysis were performed to identify hypermethylation of Formin-2 (Fmn2) promoter as the major contributor associated with the promoting effects of LDIR. To establish the role of Fmn2 in peripheral nerve regeneration, the temporal expression profile of Fmn2 after sciatic nerve crush injury was investigated, wherein PNI induced downregulation of Fmn2 expression in DRGs. Complete Fmn2 knockout (Fmn2^{-/-}) accelerated axon regeneration, and function recovery after PNI. To further determine the specific role of Fmn2 in neurons, Fmn2 expression in DRG and motor neurons was knocked down with a high neuronal tropism AAV serotype 2/9, and the specificity of neuronal knockdown was verified by eGFP-directed fluorescence activated cell sorting (FACS). Similar growth-promoting effects were observed in AAV2/9-Fmn2-shRNA mice. Mechanistically, Fmn2 knockdown increased the microtubule dynamics in growth cones of DRG neurons by enhancing the displacement rate of microtubule end-binding protein 3 (EB3) comets. In AAV2/9-Fmn2-shRNA mice, tubulin deacetylation in distal regenerating axons was increased substantially together with the upregulation of phosphorylated form of HDAC5. By inhibiting HDAC5 with a selective HDAC4/5 inhibitor LMK-235 or neuronal Hdac5 knockdown (AAV2/9-Hdac5-shRNA), the promoting effect of neuronal Fmn2 knockdown on axon regeneration was completely eliminated. Finally, a small molecule bioinformatics analysis of Fmn2^{-/-} was performed, which identified an FDA-approved small molecule, metaxalone, which promoted in vitrolin vivo axon regeneration, and function recovery. Metaxalone treatment delayed by 24 hours post-injury still maintained full therapeutic effect on function recovery.

In a first aspect, provided herein is a method of treating a nerve injury in a subject in need thereof, the method comprising: administering a therapeutically effective amount of a therapeutic agent to the subject, wherein the therapeutic agent is selected from the group consisting of metaxalone and a Formin-2 (Fmn2) inhibitor.

In certain embodiments, the Fmn2 inhibitor is an antibody, an antibody fragment, an inhibitory nucleic acid molecule, or a small molecule, wherein the inhibitory nucleic acid molecule substantially silences Fmn2.

In certain embodiments, the inhibitory nucleic acid molecule is selected from the group consisting of short interfering nucleic acid (siNA), a short interfering RNA (siRNA), a double-stranded RNA (dsRNA), a micro-RNA (miRNA), a short hairpin RNA (shRNA), a short interfering oligonucleotide, a short interfering nucleic acid, and a post-transcriptional gone silencing RNA (ptgsRNA).

In certain embodiments, the inhibitory nucleic acid molecule comprises contiguous nucleotides complementary to a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

In certain embodiments, the inhibitory nucleic acid molecule comprises SEQ ID NO: 10, SEQ ID NO:11, or SEQ ID NO:12.

In certain embodiments, administration of the inhibitory nucleic acid molecule reduces Fmn2 protein expression by 40-100%.

In certain embodiments, the therapeutic agent is administered within 24 hours of onset of the nerve injury.

In certain embodiments, the therapeutic agent is administered parenterally, paracancerally, transmucosally, transdermally, intramuscularly, intravenously, intradermally, subcutaneously, intraperitonealy, intraventricularly, intracranially, perineurally, intraneurally, or directly at a central nervous system lesion site or a peripheral nervous system lesion site comprising the nerve injury.

In certain embodiments, the inhibitory nucleic acid molecule is administered directly at a central nervous system lesion site or a peripheral nervous system lesion site comprising the nerve injury.

In certain embodiments, the therapeutic agent is metaxalone.

In certain embodiments, the nerve injury comprises at least one of a central nervous system injury or a peripheral nervous system injury.

In certain embodiments, the nerve injury is the result of physical trauma.

In certain embodiments, the nerve injury comprises an injured dorsal root ganglion.

In certain embodiments, the subject is a human, a non-human primate, an equine, a bovine, a canine, a feline, or a rodent.

In certain embodiments, the subject does not suffer from a muscle related condition.

In certain embodiments, the muscle related condition is selected from muscle spasms, muscle spasticity, and musculoskeletal pain.

In certain embodiments, nerve injury is the result of physical trauma.

In certain embodiments, administration of metaxalone begins within 24 hours of onset of the nerve injury.

In certain embodiments, metaxalone is administered orally.

In certain embodiments, the subject is a human.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1. Low dose ionizing radiation (LDIR) promotes robust axon regeneration and function recovery after peripheral nerve injury. (A) Schematic diagram illustrating the LDIR treatment paradigm for sciatic nerve pinch test. Mice were exposed to a single dose of X-ray irradiation at 2Gy immediately after the crush injury. Sciatic nerve pinch test was then performed 3 days post-injury and X-ray irradiation. (B) LD-X-ray irradiation accelerated regeneration of sensory fibers as detected by sciatic nerve pinch test, when compared with sham-irradiated controls (n=8 per group). Each dot represents the length of axonal regrowth from one mouse. (C) Representative longitudinal sections of sciatic nerve from control or X-ray-irradiated mice 3 days after crush injury immunostained with anti-SCG10 antibody, demonstrating the accelerated axon regeneration in X-ray-irradiated mice. Red dotted line indicates the site of injury and red arrowhead indicates the SCG10-positive regenerating axons at the distal nerve. Scale bar: 500 μm. (D) Regeneration index was measured as SCG10 immunoreactivity normalized to the crush site (n=4 per group). Each dot represents the normalized SCG10 immunoreactivity from one mouse. (E) Schematic diagram illustrating the treatment paradigm for sciatic nerve injury. Adult mice were exposed to a single dose of X-ray irradiation at 2Gy immediately after crush. Sensory and motor function recovery were monitored for 28 days by animal behavioral tests. (F-I) X-ray-irradiated mice exhibited substantial and long-lasting promoting effect on sensory (F) and motor (G-I) function recovery (n=8 per group). (J-K) The accelerated motor function recovery was validated by electromyography recording showing significant increase in compound muscle action potential amplitude in proximal gastrocnemius muscle (J) and distal interosseous (plantar) muscle (K) at days 7 to 21 post-injury in X-ray-irradiated mice (n=8 per group). Each dot represents the average CMAP amplitude from one mouse. Mean±SEM. *P<0.05, ***P<0.001; Student's t-test in (B), two-way ANOVA followed with post-hoc Bonferroni test in (D), or two-way ANOVA with repeated measures followed by post-hoc Bonferroni's test in (F-K).

FIG. 2. Fmn2 is a negative regulator of axon regeneration in mice. (A) Sciatic nerve transection was performed on the left sciatic nerve of adult C57BL/6 mice, and ipsilateral lumbar 4 and 5 DRGs directly supplying the sciatic nerve were harvested at days 1, 2, 3 and 5 for Fmn2 mRNA and protein expression analysis. (B-C) Fmn2 expression was significantly down-regulated in response to sciatic nerve injury at both mRNA (B) and protein (C) levels (n=3 per time point). Each dot represents one pooled mRNA or protein sample of L4/5 DRGs from 3 individual mice. FMN2 protein expression level was normalized to GAPDH. (D) Schematic diagram illustrating the paradigm for sciatic nerve pinch test in Fmn2^{-/-} mice. (E) The distal extent of sensory axonal regrowth, as measured by sciatic nerve pinch test, was markedly increased in Fmn2^{-/-} mice when compared with WT littermate mice (n=6 per group). Each dot represents the length of axonal regrowth from one mouse. (F) Representative micrographs showing that Fmn2^{-/-} mice exhibited an increased number in SCG10-positive regenerating axons distal to the crush injury site. Red dotted line indicates the site of injury and red arrowhead indicates the SCG10-positive regenerating axons at the distal nerve. Scale bar: 500 μm. (G) Regeneration index was measured as SCG10 immunoreactivity normalized to the crush site (n=4-5 per group). Each dot represents the normalized SCG10 immunoreactivity from one mouse. Mean±SEM. *P<0.05, ***P<0.001; one-way ANOVA followed with post-hoc Bonferroni test in (B and C), Student's t-test in (E), and two-way ANOVA followed with post-hoc Bonferroni test in (G). SNT, sciatic nerve transection; N, naïve control mice; WT, wild-type.

FIG. 3. Gene silencing of neuronal Fmn2 increases microtubule dynamics in cultured DRG neurons. (A) Representative photomicrographs demonstrated the growth cones of DRG neurons expressing both the microtubule plus-ends marker EB3-mCherry (red) and neuronal markers βIII-tubulin (green). Scale bar: 5 μm. (B) Series of time-lapse images indicating the movement of EB3 comets at the growing microtubule plus-ends of AAV2/9-scr-shRNA- or AAV2/9-Fmn2-shRNA-treated DRG neurons. Arrowheads with the same color indicated the movement of individual EB3 comet across time. Scale bar: 2 μm. (C) Representative kymographs of EB3 comet motion at the growing microtubule plus-ends of DRG neurons treated with AAV2/9-scr-shRNA (as control) or AAV2/9-Fmn2-shRNA. The movement of individual EB3 comets (black lines) was recorded at the rate of one frame per 2 s over a period of 5 min. Vertical scale bar: 1 min; horizontal scale bar: 1 μm. (D) The motion of each EB3 comet was automatically traced and the microtubule dynamics was assessed using plusTipTracker software. Fmn2 knockdown resulted in a substantial increase in the average microtubule growth velocity. Each dot represents the average microtubule growth velocity of EB3 comets from one growth cone. (E) Fmn2 deficiency markedly elevated the growth length of EB3 comets at the distal growing tips of DRG neurons. Each dot represents the average growth length of EB3 comets from one growth cone. (F) The microtubule growth lifetime remained unchanged after gene silencing of Fmn2. Each dot represents the average growth lifetime of EB3 comets from one growth cone. (G) Gene silencing of Fmn2 did not alter the EB3 comet density at the growing microtubule plus-ends of DRG neurons. Each dot represents the average density of EB3 comets from one growth cone. A total of 31 (AAV2/9-scr-shRNA-treated neurons) and 39 (AAV2/9-Fmn2-shRNA-treated neurons) growth cones were analyzed from 3 independent experiments. Mean±SEM. **P<0.01, ***P<0.001; Mann-Whitney U test in (D-G). n.s., not significant.

FIG. 4. Fmn2 expression is a critical determinant of α-tubulin deacetylation in the regenerating axons in a HDAC5-dependent manner. (A) To knockdown the gene expression of Fmn2 and Hdac5 in DRGs, AAV2/9-Fmn2-shRNA, AAV2/9-Hdac5-shRNA or AAV2/9-scrambled (scr)-shRNA were injected directly to the sciatic nerves two weeks before sciatic nerve crush injury. Selective blockade of HDAC5 was achieved by injecting LMK-235 (20 mg/kg) intraperitoneally immediately after crush for 3 consecutive days. Sciatic nerve pinch test and axon quantifications were performed three days after sciatic nerve crush injury to assess the distal extent of axon regeneration. (B) Compared with AAV2/9-scr-shRNA mice, the distal extent of axonal regrowth was markedly increased in AAV2/9-Fmn2-shRNA mice. Pharmaceutical blockade of HDAC5 activity using LMK-235 or gene silencing of HDAC5 using AAV2/9-Hdac5-shRNA markedly reduced the growth-promoting effects induced by Fmn2-deletion (n=6 per group). Each dot represents the length of axonal regrowth from one mouse. (C) Representative SCG10 immunostaining of sciatic nerves with different AAV2/9-shRNA and LMK-235 treatments 3 days after crush injury. Red dotted line indicates the site of injury and red arrowhead indicates the SCG10-positive regenerating axons at the distal nerve. Scale bar: 500 μm. (D) SCG10 immunoreactivity was measured at different distal distances and normalized to the level in the crush site as the regenerative index (n=4 per group). Each dot represents the normalized SCG10 immunoreactivity from one mouse. (E) At 3 days post-injury, representative immunoblots revealed the activation of HDAC5 phosphorylation associated with reduction of acetylated tubulin in the regenerating axons of AAV2/9-Fmn2-shRNA mice. Both pharmaceutical blockade and knockdown of HDAC5 eliminated the Fmn2-deletion induced tubulin deacetylation at the growing axons. (F) Bar graph showed normalized fold change of pHDAC5 protein expression. pHDAC5 protein expression levels were normalized to GAPDH. Each dot represents one pooled protein sample of segmented sciatic nerves (0-6 mm distal to the crush site) from 3 individual mice. (G) Bar graph showed normalized fold change of acetylated tubulin/total tubulin protein expression. Acetylated tubulin protein expression levels were first normalized to total tubulin levels, and then normalized to GAPDH expression. Each dot represents one pooled protein sample of segmented sciatic nerves (0-6 mm distal to the crush site) from 3 individual mice. Mean±SEM. *P<0.05 when compared with AAV2/9-scr-shRNA-treated control mice; #P<0.05 when compared with AAV2/9-Fmn2-shRNA-treated mice; one-way ANOVA, followed by post-hoc Bonferroni test in (B, E-F), two-way ANONA followed with post-hoc Bonferroni test in (D).

FIG. 5. Fmn2 deletion induces distinct transcriptional reprogramming as a unique gene signature for in silico screening of FDA-approved small molecules. L4/5 DRGs were used for RNA-seq whole transcriptome analysis 5 days after sciatic nerve injury. (A-B) Hierarchical clustering and principal component analysis showed a clear separation between naïve (uninjured) and injured DRG neurons regardless of genotypic differences. (C) A total of 397 genes were up-regulated in both injured wildtype (WT) and Fmn2--DRGs, which represented the common core transcriptional change in both genotype groups in response to injury. There were 87 up-regulated and 58 down-regulated genes that were differentially expressed in injured DRGs of Fmn2^{-/-} mice only, which represented genotype-specific transcriptional regulation caused by genetic ablation of Fmn2. (D) Heatmap of differentially expressed genes in which a total of 145 genes were differentially expressed only in injured DRGs of Fmn2^{-/-} mice. Color intensity represents changes in log2FC from negative (light blue) to positive (dark brown).

FIG. 6. Clinical relevance FDA-approved metaxalone promotes in vivo axon regeneration after PNI. Whole transcriptome analysis and small molecule bioinformatics analysis identified four top-ranked bioactive small molecules for testing. (A) Schematic diagram illustrating administration of small molecules directly to the site of injury immediately after the sciatic nerve crush for 3 consecutive days (twice per day with an 8-hour time interval). Sciatic nerve pinch test was performed 72 hours after injury. (B) FDA-approved small molecule metaxalone induced robust in vivo axon regeneration (n=4 per group). Each dot represents the length of axonal regrowth from one mouse. (C) Micrographs of the longitudinal sections of sciatic nerves showed higher number of SCG10-positive regenerating axons in metaxalone-treated mice distal to the crush injury site, when compared with the vehicle controls. (n=4 per group). Red dotted line indicates the site of injury and red arrowhead indicates the SCG10-positive regenerating axons at the distal nerve. Scale bar: 500 μm. (D) Regeneration index was measured as SCG10 immunoreactivity normalized to the crush site. Each dot represents the normalized SCG10 immunoreactivity from one mouse. (E) Representative immunoblots demonstrated that metaxalone treatment induced robust activation of HDAC5 phosphorylation and reduced expression of acetylated tubulin in sciatic nerves at 3 days post-injury. (F) Bar graph showed normalized fold change of pHDAC5 protein expression. pHDAC5 protein expression levels were normalized to GAPDH. Each dot represents one pooled protein sample of segmented sciatic nerves (0-6 mm distal to the crush site) from 3 individual mice (n=3 per group). (G) Bar graph showed normalized fold change of acetylated tubulin/total tubulin protein expression. Acetylated tubulin protein expression levels were first normalized to total tubulin levels, and then normalized to GAPDH expression. Each dot represents one pooled protein sample of segmented sciatic nerves (0-6 mm distal to the crush site) from 3 individual mice (n=3 per group). Mean±SEM. *P<0.05, **P<0.01, ***P<0.001; one-way ANOVA followed with post-hoc Bonferroni test in (B), two-way ANOVA followed with post-hoc Bonferroni test in (D), Student's t-test in (F and G).

FIG. 7. Delay metaxalone treatment still promotes robust in vivo axon regeneration and function recovery after PNI. (A) Schematic diagram illustrating the experimental paradigm for delayed metaxalone treatment. Metaxalone (0.25 μg/μl) was administrated to the site of injury one day after sciatic nerve crush for 2 consecutive days (twice per day with an 8-hour time interval). Sciatic nerve pinch test was performed at day 3 post-injury to assess the extent of distal sensory axonal regrowth. (B) Delayed metaxalone treatment by 24 h was as effective as regular metaxalone treatment in promoting in vivo axon regeneration (n=5 per group). Each dot represents the length of axonal regrowth from one mouse. (C) Representative confocal micrographs demonstrated that delayed metaxalone treatment regrew more and longer SCG10-positive regenerating axons than vehicle controls. Red dotted line indicates the site of injury and red arrowhead indicates the SCG10-positive regenerating axons at the distal nerve. Scale bar: 500 μm. (D) Regeneration index was measured as SCG10 immunoreactivity normalized to the crush site (n=4 per group). Each dot represents the normalized SCG10 immunoreactivity from one mouse. (E) Immediately after sciatic nerve crush injury, adult mice were injected intraperitoneally with metaxalone (10 mg/kg) for 21 consecutive days or delaying daily intraperitoneal injection of metaxalone (10 mg/kg) to 24 hours post-injury for 20 consecutive days. Animal behavioral assessments and weekly EMG recordings were performed. (F-I) Both metaxalone-treated and delayed-metaxalone-treated mice displayed acceleration of sensory (F) and motor (G-I) function recovery to similar extents, when compared with the vehicle controls (n=8-11 per group). (J-K) Improved motor function recovery in metaxalone-treated and delayed-metaxalone-treated mice were confirmed by significant increase in CMAP amplitudes in proximal (gastrocnemius; J) and distal plantar (interosseous; K) muscles at days 7 to 21 post-injury compared with vehicle controls. (n=7-8 per group). Each dot represents the average CMAP amplitude from one mouse. Mean±SEM. *P<0.05, **P<0.01, ***P<0.001; Student's t-test in (B), two-way ANONA followed with post-hoc Bonferroni test in (D), two-way ANOVA with repeated measures, followed by Bonferroni's post hoc test in (F-K).

FIG. 8. Low-dose ionizing radiation enhances axonal regrowth of axotomized DRG neurons in cultures. (A) Schematic diagram illustrating α-particle treatment paradigm for DRG cultures. A 3.5 μm-thick Mylar film was cut at a size of 18×18 mm and glued onto the bottom of a 9 mm hole located at the center of a 35 mm petri dish. A total of 2,000 DRG neurons were plated onto the Mylar film (yellow highlighted area) pre-coated with poly-D lysine (100 μg/ml) and laminin (10 μg/ml) as indicated by black arrowheads. A planar 241 Am α-particle source was placed under a 9 mm-hole covered by Mylar film. DRG neurons received a single exposure to α-particle irradiation (green arrowhead) at different doses, and allowed to grow for 17 h for neurite outgrowth assays. (B) DRG neurons immunostained with anti-βIII-tubulin antibodies exhibited significantly longer neurites following a single exposure to α-particle irradiation at 20mGy, when compared with other treatment groups. Each dot represents the total neurite length or average longest neurite from one independent cell culture experiment. Scale bar: 500 μm. (C) DRG neurons were placed at the centre of the X-ray irradiator and exposed to a single dose of X-ray irradiation, using a cabinet irradiator X-Rad 320 (Precision X-Ray) and allowed to grow for 17 h for neurite outgrowth assay. (D) DRG neurons received a single exposure to X-ray irradiation at 150mGy exhibited significantly longer neurites when compared with sham-irradiated controls. Each dot represents the total neurite length or average longest neurite from one independent cell culture experiment. Scale bar: 500 μm. (E) Both α-particle and X-ray irradiation did not affect the cell survival of cultured DRG neurons. Each dot represents the mean value of one independent cell culture experiment. (F) Schematic diagram illustrating the LDIR treatment paradigm for ex vivo DRG explant culture. Scale bar: 500 μm. (G) Whole-body exposure to X-ray irradiation at 2Gy induced maximal promoting effects on neurite outgrowth from axotomized DRG explants (n=5 DRG explants per group from 3 independent experiments). Scale bar: 500 μm. Each dot represents the total neurite outgrowth or average longest neurite from one DRG. Mean±SEM of triplicates. *P<0.05, **P<0.01; one-way ANOVA followed by post hoc Bonferroni test. n.s., no significant difference; CTL, control.

FIG. 9. Alpha-particle and X-ray irradiation induce gene-specific DNA methylation at the CpG sites of promoter regions without affecting the global DNA methylation and key regeneration-associated genes (RAGs) expression in DRG neurons. DRG neurons were subjected to α-particle (20mGy) and X-ray (150mGy) irradiation, allowed to grow for 17 hours, and genomic DNA were extracted. A genome-wide reduced representation bisulfite sequencing (RRBS) was performed. (A) Global DNA methylation in DRG neurons was not affected by either α-particle or X-ray irradiation. (B) Alpha-particle and X-ray irradiation did not alter the gene expression of DNA methylation machinery. Each dot represents the mRNA expression from one independent cell culture experiment. (C) No significant differences in gene expression levels of key RAGs were observed in DRG neurons after treated with either α-particle- or X-ray-irradiation. Each dot represents the mRNA expression from one independent cell culture experiment. (D) The number of genes with significant hypermethylation and hypomethylation at the CpG sites of promoter regions in axotomized DRG neurons after exposure to α-particle irradiation at 20mGy. (E) In the X-ray-irradiated DRG neurons, a total of 124 genes (44 hypomethylated+80 hypermethylated) showed differential methylation at the CpG sites of promoter regions, when compared with sham-irradiated controls. Mean±SEM of triplicates. P>0.05, two-way ANONA, followed by post hoc Bonferroni test.

FIG. 10. Hypermethylation of Fmn2 promoter is correlated with a significant downregulation of Fmn2 transcripts. Primary dissociated cultures of DRG neurons were subjected to 20mGy of α-particle or 150mGy of X-ray irradiation. Irradiated neurons were cultured for 17 hours and total RNA were extracted and quantitative polymerase chain reaction analysis was performed to examine transcriptional changes. Of these seven candidate genes, only Fmn2 showed significant down-regulation in both LDIR-treated cultures, which was well correlated with the hypermethylation of CpG dinucleotides at its promoter region (n=4 per group). Each dot represents the mRNA expression from one independent cell culture experiment. Mean±SEM. *P<0.05, Kruskal-Wallis test, followed by Dunn's post hoc test.

FIG. 11. Genetic ablation of Fmn2 potentiates the intrinsic growth capacity of preconditioned DRG neurons. (A) FMN2 protein was abundantly expressed in axotomized DRG neurons prepared from WT mice. However, no FMN2 immunoreactivity was detected in DRG neurons of Fmn2−/− mice. DRG neurons prepared from Fmn2−/− mice exhibited robust neurite outgrowth (n=3 per group). Scale bar: 500 μm. Each dot represents the total neurite outgrowth or average longest neurite from one independent cell culture. (B) Significant increased neurite outgrowth in contralateral DRGs from Fmn2−/− mice was observed. Ablation of Fmn2 further potentiated the preconditioning effects in neurite outgrowth (n=8-11 DRG explants per group from 3 independent experiments). Each dot represents the total neurite outgrowth or average longest neurite from one DRG. Scale bar: 500 μm. Mean±SEM. *P<0.05, when compared with WT controls; #P<0.05 when compared with pre-conditioned (ipsilateral) WT DRG explants; Student's t-test in (A), two-way ANOVA followed by posthoc Bonferroni test in (B). WT, wild-type.

FIG. 12. Genetic ablation of Fmn2 promotes sensory and motor function recovery after sciatic nerve injury. (A) Sensory function recovery, as assessed by response to pinprick stimulation, was significantly accelerated in Fmn2−/− mice at days 7-15 post-injury. (B) Fmn2−/− mice exhibited a better recovery of toe spreading reflex motor function at days 11-21 postinjury. (C) Hindlimb grip strength of Fmn2−/− mice was significantly higher than the controls at days 7-23 post-injury. (D) Fmn2−/− mice demonstrated improved sciatic functional index (SFI) over the course of recovery. (E-F) CMAP amplitudes of gastrocnemius and interosseous muscles were significantly higher in Fmn2−/− mice than their age-matched WT littermates at days 7-21 post-injury. Each dot represents the average CMAP amplitude from one mouse. Mean±SEM (n=8 11 per group). *P<0.05; two-way repeated measures ANOVA, followed by Bonferroni's post hoc test. WT, wild-type.

FIG. 13. Adeno-associated virus 2/9 (AAV2/9)-based gene delivery system shows preferential transduction of DRG and motor neurons after sciatic nerve injection. (A) Representative fluorescence micrographs demonstrated preferential transduction of AAV2/9-scr-shRNA (tagged with eGFP) in DRG neurons 2 weeks after sciatic nerve injection. Approximately 72% of the total βIII-tubulin-positive DRG neurons were GFP-positive (n=5 per group). Each dot represents the percentage of GFP-and βIII-tubulin-positive DRG neurons from one mouse. Scale bar: 100 μm. (B) Representative confocal micrographs demonstrated that AAV2/9 scr-shRNA (tagged with eGFP) successfully transduced the ChAT-positive motor neurons in the ventral horns. Scale bar: 100 μm. Approximately 75% of the total ChATpositive motor neurons were GFP-positive (n=5 per group). Each dot represents the percentage of GFP-and ChAT-positive motor neurons from one mouse. (C) The immunoreactivity of GFP showed minimal overlapping with SOX10 positive Schwann cells. Scale bar: 100 μm. (D) Representative confocal micrographs demonstrated that AAV2/9-scrshRNA (tagged with eGFP) did not transduce SOX10-positive Schwann cells near the injection site of the sciatic nerves. Scale bar: 100 μm. (E) The immunoreactivity of GFP showed virtually no overlapping with CD68-positive macrophages. Scale bar: 100 μm.

FIG. 14. Adeno-associated virus 2/9 (AAV2/9)-based gene delivery system efficiently transduces the DRG and motor neurons after sciatic nerve injection. (A-B) Representative FACS plots showing the gating strategies to isolate the GFP-positive cells from DRGs for Western blot analysis. After enzymatic and mechanical dissociation of DRGs, the cell suspension was first gated based on the cell size (forward scatter; FSC-A, x-axis) and surface characteristics (side scatter; SSC-A, y-axis) (left panel in A). Next, single cells were selected and aggregated cells were discarded based on the ratio of FSC-A and FSC-H (right panel in A). The dissociated cells from untransduced L4/5 DRG neurons (i.e. with no GFP expression) were used as negative controls (left panel in B) to set up the threshold of FITC-A (x-axis representing the fluorescence intensity of GFP) for the selection of GFP-positive cells. The GFP-positive cells within the gate (yellow inset of the right panel in B) were collected for subsequent Western blot analysis. (C) The sorted GFP-positive cells expressed a high level of NeuN (a neuronal marker) and GFP (the fluorescence tag), and virtually no expression of major glial cell markers such as SOX10 (a marker for Schwann cells) and IBA-1 (a marker for microglia), suggesting preferential transduction of DRG neurons rather than non-neuronal cells after injection of AAV2/9 into the sciatic nerves. Compared with AAV2/9-scr-shRNA controls, the sorted GFP-positive cells from AAV2/9-Fmn2-shRNA mice expressed a significantly lower level of FMN2 protein (n=3 per group). Each dot represents one protein sample of sorted GFP-positive cells pooled from 3 individual mice. (D) Western blot analysis revealed a significant down-regulation of FMN2 protein expression in the lumbar ventral horn of spinal cord from AAV2/9-Fmn2-shRNA-treated mice. Each dot represents one pooled protein sample of ventral horn from 3 individual mice. Mean±SEM. *P<0.05; Student's t-test in (C and D). (E) In AAV2/9-Fmn2-shRNA-treated mice, FMN2 protein expression was significantly reduced in the transduced GFP-positive and ChAT-positive motor neurons (white arrowheads). The untransduced non-GFP ChAT-positive motor neurons (yellow arrowheads) showed high immunoreactivity of FMN2 protein expression. Scale bar: 100 μm.

FIG. 15. AAV2/9-mediated gene silencing of Fmn2 in DRGs accelerates in vivo axon regeneration, and promotes sensory and motor functional recovery after sciatic nerve crush injury in adult mice. (A) To knockdown the gene expression of Fmn2 in DRGs, AAV2/9-Fmn2-shRNA or AAV2/9-scrambled (scr)-shRNA were injected directly to the sciatic nerves two weeks before sciatic nerve crush injury. Axon quantifications were performed three days after sciatic nerve crush injury to assess the distal extent of axon regeneration. (B) Sciatic nerve injection of AAV2/9-Fmn2-shRNA markedly reduced the FMN2 protein expression in L4/5 DRGs directly supplying the sciatic nerve two weeks after injection (n=3 per group). Each dot represents one pooled protein sample of L4/5 DRGs from 3 individual mice. (C-D) Representative confocal micrographs showed that a substantial higher level of SCG10 immunoreactivity were detected at the distal axonal segment of injured sciatic nerve from AAV2/9-Fmn2-shRNA mice. SCG10 immunoreactivity was measured at different distal distances and normalized to the level in the crush site as the regenerative index (n=4 per group). The quantification data of SCG10 immunoreactivity for both AAV2/9-scr-shRNA and AAV2/9-Fmn2-shRNA mice was extracted from FIG. 4C. Each dot represents the normalized SCG10 immunoreactivity from one mouse. Red dotted line indicates the site of injury and red arrowhead indicates the SCG10-positive regenerating axons at the distal nerve. Scale bar: 500 μm. (E) To in vivo knockdown Fmn2 gene expression in DRGs, AAV2/9-Fmn2-shRNA was injected directly into the left sciatic nerve of adult male C57BL/6 mice two weeks before sciatic nerve crush injury. AAV2/9-scrambled (scr)-shRNA was used as a control. Sensory and motor functional recovery were monitored for 29 days by an exhaustive list of animal behavioral tests. (F-I) AAV2/9-Fmn2-shRNA-treated mice showed improved functional recovery on sensory (F) and motor (G-I) functions, compared with AAV2/9-scrshRNA-treated controls. (J-K) The accelerated motor functional recovery in AAV2/9-Fmn2-shRNA-treated mice was further confirmed by a significant elevation in CMAP amplitudes in proximal (gastrocnemius; F) and distal plantar (interosseous; G) muscles at days 7 to 21 postinjury compared with AAV2/9-scr-shRNA-treated controls. Each dot represents the average CMAP amplitude from one mouse (n=7-8 per group in F-K). Mean±SEM. *P<0.05; Student's t-test in (B), two-way ANOVA followed with post-hoc Bonferroni test in (D), or two-way ANOVA with repeated measures, followed by post-hoc Bonferroni's test in (F-K).

FIG. 16. Efficient gene silencing of HDAC5 proteins in lumbar 4 and 5 (L4/5) DRGs by sciatic nerve injections of AAV2/9-Hdac5-shRNA. Sciatic nerve injection of AAV-Hdac5-shRNA significantly reduced the protein expression of HDAC5 in L4/5 DRGs two weeks after injection. Each dot represents one pooled protein sample of L4/5 DRGs from 3 individual mice. Mean±SEM of triplicate. *P<0.05; Student's t-test.

FIG. 17. Fmn2-deletion induces up-regulation of regeneration-associated genes at levels similar to wildtype mice and activates inflammatory responses of injured DRGs. (A) Core regeneration-associated genes were induced in both injured WT and Fmn2−/− DRGs, suggesting that both genotypes shared a common core transcriptional reprogramming in response to injury (n=4 per group). Each dot represents one pooled mRNA sample of L4/5 DRGs from 3 individual mice. Mean±SEM. *P<0.05 when compared with WT naive (i.e. uninjured) DRGs; Kruskal Wallis test, followed by Dunn's post hoc test. (B) We applied gene set enrichment analysis to a total of 145 genes that were differentially expressed only in injured DRGs of Fmn2−/− mice and identified core gene regulatory network such as Jak-Stat and chemokine/cytokine signalling pathways.

FIG. 18. Metaxalone enhances the intrinsic growth capacity of axotomized DRG neurons. (A) DRG neurons immunostained with anti-βIII-tubulin antibodies exhibited extensive neurite outgrowth after treating with increasing dose of metaxalone and neurite outgrowth was increased almost linearly until reaching a plateau at 20 μM. Scale bar: 500 μm. (B-C) The total neurite outgrowth (B) and the longest neurite length (C) of DRG neurons were averaged from at least 250 neurons per treatment group in one independent cell culture experiment (n=4 per group). Each dot represents the total neurite length or average longest neurite from one independent cell culture experiment. Mean±SEM. *P<0.05; one-way ANOVA followed with post-hoc Bonferroni test in (B and C).

FIG. 19. The percentage change of DNA methylation levels of the seven candidate genes at their promoter regions in DRG neurons after LDIR of alpha-particle (20mGy) and X-ray (150mGy). Positive and negative values indicated hypermethylation and hypomethylation, respectively.

FIG. 20. Top-ranked bioactive small molecules with connectivity scores over 95 were shortlisted for functional assessment using sciatic nerve pinch test. Metaxalone and acamprosate are FDA-approved drugs.

FIG. 21. The sequence of qPCR primers used herein.

FIG. 22. Sequences of the shRNAs targeting mouse Fmn2 and Hdac5 mRNA transcripts and a non-targeting scrambled sequence for AAV vector construction.

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”. “including”, and the like; and that terms such as “consisting essentially of” and “consists essentially of”' bave the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “subject” refers to any animal (e.g., a mammal), including, but not limited to, humans, non-human primates, canines, felines, and rodents.

As used herein the term “substantially silences Fmn2” means that expression or protein activity or level of the target gene or protein encoded by the target gene in the presence of the introduced inhibitory nucleic acid molecule targeting a Fmn2 nucleic acid sequence is reduced by about 10% to 100%, 10% to 90%, 20% to 90%, 30% to 90%, 40% to 90%, 50% to 90%, 50% to 80%, 50% to 70%, 55% to 70%, SS% to 69.2%, 70% to 90%, or 80% to 90% as compared to the expression or protein activity or level of the target gene or protein encoded by the target gene seen when the inhibitory nucleic acid molecule targeting a Formin-2 nucleic acid sequence is not present. Generally, when a gene is substantially silenced, it will have at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or even 100% reduction in expression or protein activity or level of the target gene or protein encoded by the target gene as compared to when the inhibitory nucleic acid molecule is not present.

The term “therapeutically effective amount” as used herein, means that amount of the compound or therapeutic agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

The present disclosure provides a method of treating a nerve injury in a subject in need thereof, the method comprising: administering a therapeutically effective amount of a therapeutic agent to the subject, wherein the therapeutic agent is selected from the group consisting of metaxalone and a Fmn2 inhibitor.

In certain embodiments in which the therapeutic agent is metaxalone, the subject does not suffer from a muscle related condition. The term “musculoskeletal condition” can include any condition affecting the muscles, tendons, ligaments, bones, joints, and associated tissues that move the body and maintain its form. Such conditions include conditions that can originate in the muscles, tendons, ligaments, or bones and associated tissues or conditions that originate elsewhere in the body, for example in the central or peripheral nervous system, that are manifested in the muscles, tendons, ligaments, bones, joints or associated tissues. In certain embodiments, the muscle related condition is selected from muscle spasms, muscle spasticity, and muscular and/or skeletal pain.

The Fmn2 inhibitor can be an antibody, an antibody fragment, an inhibitory nucleic acid molecule, a small molecule (synthetic or naturally isolated or derived) a protein, a peptide, carbohydrate or an antibody, wherein the inhibitory nucleic acid molecule substantially silences Fmn2.

The antibody can be a monoclonal antibody or a polyclonal antibody. The antibody may be a single chain antibody. The antibody can be a chimeric antibody. The antibody can be a human antibody or a humanized antibody.

The inhibitory nucleic acid molecule can be a short interfering nucleic acid (siNA), a short (siRNA), a double-stranded RNA (dsRNA), a micro-RNA (miRNA), a short hairpin RNA (shRNA), a short interfering oligonucleotide, a short interfering nucleic acid, or a post-transcriptional gene silencing RNA (ptgsRNA).

Generally, an inhibitory nucleic acid molecule can be unmodified or modified. In certain embodiments, an inhibitory nucleic acid molecule comprises one or more modified oligonucleotides, e.g., phosphorothioate-, 2′-O-methyl-, and the like modified oligonucleotides, are known in the art to improve the stability of oligonucleotides in vivo.

In certain embodiments, the inhibitory nucleic acid molecule comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding Fmn2, such as SEQ ID NO:1, SEQ ID NO:2, SEQ ID NO:3, SEQ ID NO:4, SEQ ID NO:5, SEQ ID NO: 6, SEQ ID NO:7, SEQ ID NO:8, and SEQ ID NO:9. In certain embodiments, the inhibitory nucleic acid molecule comprises 15-50 nucleotides, 15-45 nucleotides, 15-40 nucleotides, 15-35 nucleotides, 15-30 nucleotides, 15-25, or 20-29 nucleotides, and can include a 3′ and/or 5′ overhang on each strand having a length of about 0, 1, 2, 3, 4, or 5 nucleotides. Inhibitory nucleic acid molecules useful in the method described herein also include sequences that bind under stringent conditions to nucleic acids that are 80%, 85%, 90%, or 95% homologous to 5 and 50 continuous nucleotides in NM_001305424.2 (SEQ ID NO:1), NM_020066.5 (SEQ ID NO: 2), NM_001348094.2 (SEQ ID NO:3), XM_017001837.2 (SEQ ID NO:4), XM_017001838.2 (SEQ ID NO:5), XM_011544237.4 (SEQ ID NO:6), XM_047425620.1 (SEQ ID NO:7), XM_017001840.3 (SEQ ID NO:8), and XM_017001841.3 (SEQ ID NO:9).

In certain embodiments, the inhibitory nucleic acid molecule comprises SEQ ID NO: 10, SEQ ID NO:11, or SEQ ID NO:12. In certain embodiments, the inhibitory nucleic acid molecule is a shRNA comprising SEQ ID NO: 10, SEQ ID NO: 11, or SEQ ID NO:12.

The nerve injury can comprise an injury to the central nervous system or the peripheral nervous system.

The nerve injury can be acute or chronic. A nerve injury can comprise the complete severing or partial severing of a neuron or crushing or compression injury to a neuron. In certain embodiments, the nerve injury directly impairs the normal functioning of neuron(s). In certain embodiments, the nerve injury indirectly impairs the normal functioning of the neuron(s). The nerve injury can result from an acute or traumatic event, chronic event, pressure build-up, or chronic neurodegeneration. Injuries to a subject can result in injury to a neuron. Common causes of nerve injuries include, but are not limited to, disease and/or infection, ischemia, anoxia, hypoglycemia, contusion, laceration, trauma to the brain or spinal cord (such as caused by acute spinal cord damage or stroke), damage by exogenous chemical agents, and combinations thereof.

The nerve injury can be the result of a disease, disorder, or condition in a subject, such as damage to a dorsal root ganglion; traumatic brain injury; stroke related injury; a cerebral aneurism related injury: a spinal cord injury, including monoplegia, diplegia, paraplegia, hemiplegia and quadriplegia; a neuroproliferative disorder or neuropathic pain syndrome.

In certain embodiments, the subject that suffers from a nerve injury resulting from a trauma. The nerve injury may comprise injury to the optic nerve, the spinal cord, or a peripheral nerve injury. In certain embodiments, the spinal cord injury or peripheral nerve injury comprises an injured dorsal root ganglion neuron.

In certain embodiments, the subject suffers from a disease or condition that results in nerve injury. In certain embodiments, the disease or condition is selected from the group consisting of stroke, spinal cord injury, Huntington's disease, Parkinson's disease, Alzheimer's disease, multiple system atrophy, spino-cerebellar atrophy, motor neuropathy, epilepsy or seizures, peripheral neuropathy, cerebral palsy, glaucoma, age related loss of neurons or neuronal connectivity and related deterioration of sensory, motor, reflect, and cognitive abilities.

In certain embodiments, the subject that suffers from an injury caused by or associated with peripheral neuropathies, such as diabetic neuropathy, virus-associated neuropathy, botulism-related neuropathy; toxic polyneuropathy, nutritional neuropathy, angiopathic neuropathy, sarcoid-associated neuropathy; carcinomatous neuropathy, compression neuropathy, and/or hereditary neuropathy; and/or peripheral nerve damage associated with spinal cord injury.

Specific routes of administration and the dosage regimen of the therapeutic agent can be determined by skilled clinicians, based on factors such as the exact nature of the condition being treated, the severity of the condition, and the age and general physical condition of the patient.

In certain embodiments, the therapeutic agent is administered parenterally, paracancerally, transmucosally, transdermally, intramuscularly, intravenously, intradermally, subcutaneously, intraperitonealy, intraventricularly, intracranially, perineurally, intraneurally, or directly at a central nervous system lesion site or a peripheral nervous system lesion site comprising the nerve injury. In instances in which the therapeutic agent is metaxalone or a small molecule, the therapeutic agent can be administered orally.

The exact dosage amount of a therapeutic agent described can vary according to factors known in the art including, but not limited to, the physical/chemical nature of the compound, the properties of the pharmaceutically acceptable carrier, the intended dosing regimen, the state of the subject's nervous system injury, and the method of administering the compound. Those of ordinary skill in the art, however, can readily determine the appropriate amount with consideration of such factors.

In certain embodiments, the therapeutic agent is administered to the subject within 1 hour, 2 hours, 3 hours, 4 hours, 5 hours, 6 hours, 12 hours, 24 hours, 36 hours, 48 hours, 60 hours, or 72 hours of onset of the nerve injury.

### DETAILED DESCRIPTION

### Results

### LDIR Enhances the Intrinsic Growth Capacity of Injured Neurons and Functional Recovery

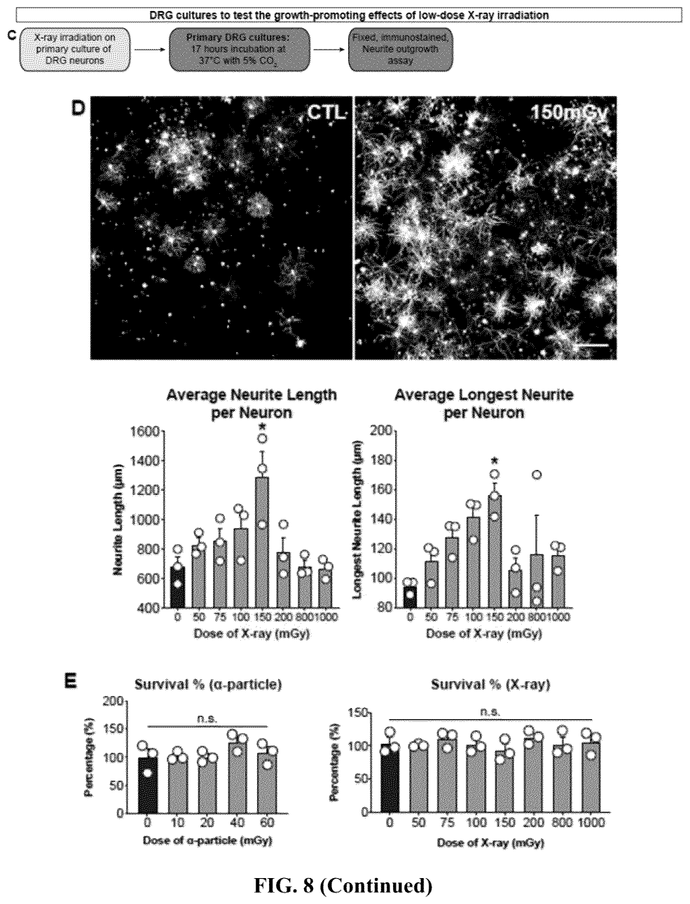

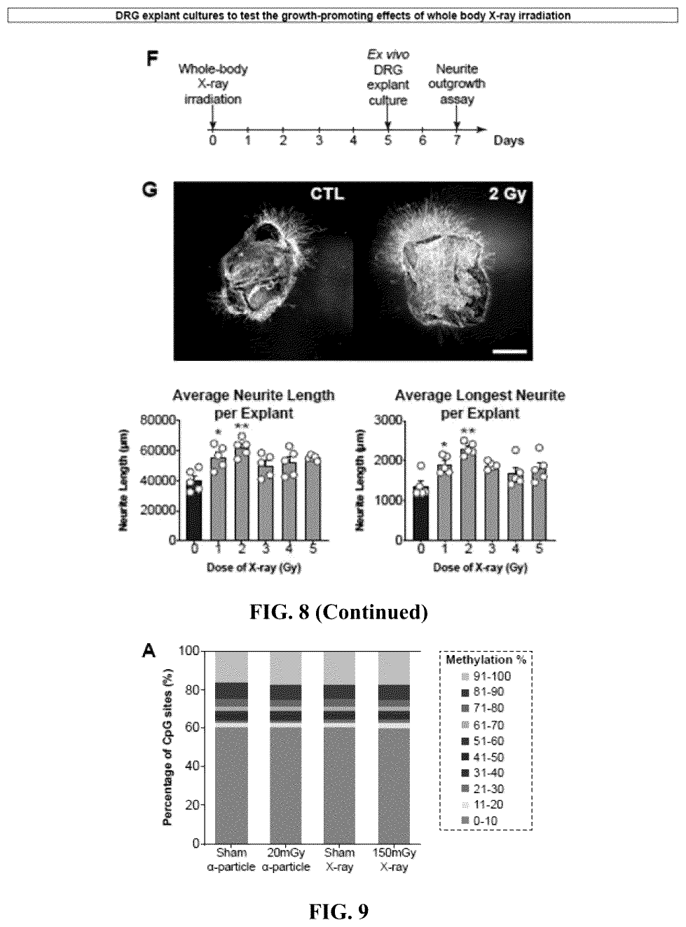

Ionizing radiation such as α-particle and X-ray are commonly used in biological studies and medical applications. LDIR exerts neuroprotective effects in mouse models of Alzheimer's disease, Parkinson's disease, glaucoma, diabetic neuropathy, and retinitis pigmentosa. In a recent study, the beneficial effect of LDIR was observed in rats by performing a local irradiation on sciatic nerve directly, but the underlying mechanism remains unknown. It was therefore hypothesize that LDIR not only promotes the overall physiological functioning of living organisms but also enhances intrinsic growth capacity of injured DRG neurons since DRGs directly supply the sciatic nerve. It was first demonstrated that 20mGy α-particle induced 39.9% and 35.5% increase in total neurite outgrowth and average longest neurite length of DRG neurons, respectively (FIG. 8A-B). Furthermore, a robust neurite outgrowth after LD X-ray irradiation was observed, which has a higher penetrating power than α-particle, with its maximal promoting effect on total neurite outgrowth (89.0%) and longest neurite length at 150mGy (66.7%) (FIG. 8C-D). Alpha-particle (α-particle) and X-ray irradiation showed no adverse effects on cell survival as measured with the WST-1 cell cytotoxicity assay (FIG. 8E).

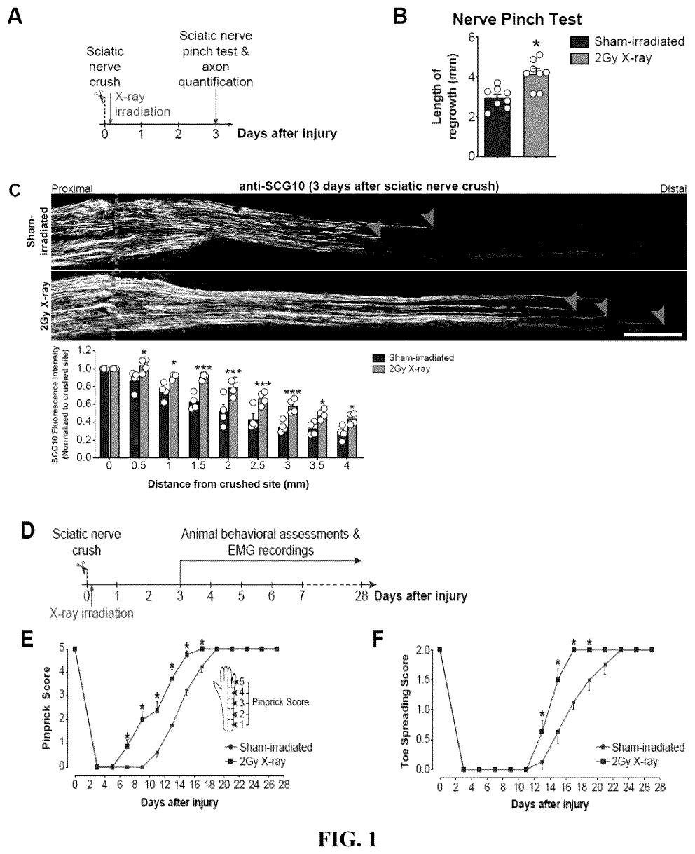

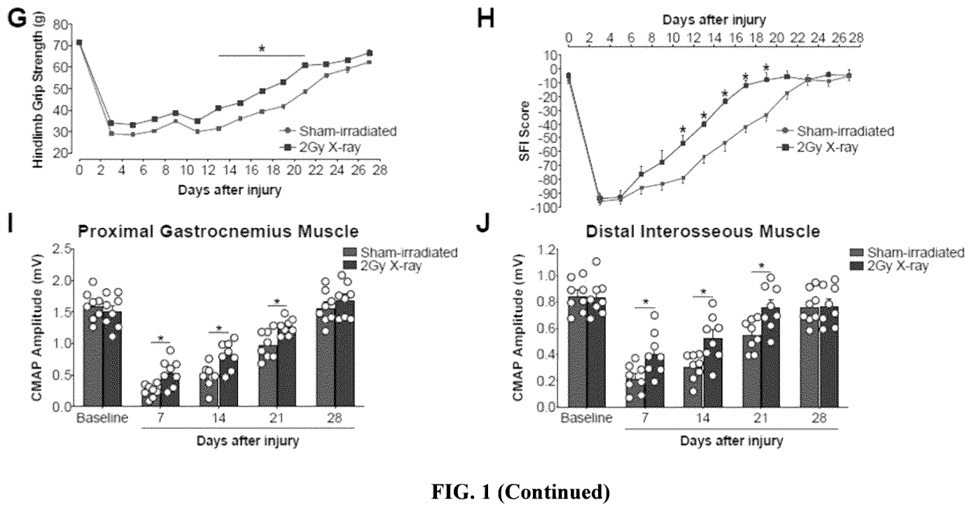

These in vitro findings were then extended to whole-body X-ray irradiation (FIG. 8F) and it was investigated whether LD X-ray irradiation could increase the intrinsic growth capacity of axotomized neurons in DRG explant cultures. Notably, whole-body X-ray irradiation at 2Gy showed a dramatic increase of 54.8% and 68.4% in total neurite outgrowth and longest neurite length, respectively (FIG. 8G). The ex vivo DRG explant results were validated with sciatic nerve pinch test to measure the rate of in vivo axon regeneration (FIG. 1A). A 41.2% increase in axon regeneration in mice exposed to a single X-ray irradiation at 2Gy immediately after crush was observed, when compared with sham-irradiated mice (FIG. 1B). The nerve pinch test results were validated by immunostaining for SCG10, which is preferentially expressed in regenerating sensory axons. Regeneration index was calculated by normalizing the average SCG10 immunoreactivity in the longitudinal section of sciatic nerve measured at different distal distances to the SCG10 immunoreactivity at the crush site, indicating the length and the number of axons have regenerated past the crush site (FIG. 1C). The regeneration index at the region over 4 mm from the injury site was significantly higher in nerves treated with 2Gy X-ray irradiation compared with sham-irradiated control 3 days after sciatic nerve crush, demonstrating that X-ray irradiation accelerates axon regeneration in the injured sciatic nerves of mice (FIG. 1D).

To examine if this increase in intrinsic growth capacity could promote in vivo functional recovery for an extend period of time (28 days), adult mice were exposed to a single dose of X-ray irradiation at 2Gy immediately after crush. Sensory and motor function recovery were monitored for 28 days by a battery of animal behavioral tests (FIG. 1E). X-ray-irradiated mice demonstrated an early initial pinprick sensory response, which was 4 days earlier than the sham-irradiated mice (FIG. 1F). For motor function recovery, X-ray-irradiated mice regained full toe spreading reflex 6 days earlier than sham-irradiated mice (FIG. 1G) and showed a faster recovery of hindlimb grip strength than the sham-irradiated mice (FIG. 1H). Gait analysis of walking track by sciatic function index (SFI) demonstrated that at days 11-19 post-injury, the motor recovery of X-ray-irradiated mice was significantly higher than the sham-irradiated mice (FIG. 1I). The accelerated motor function recovery was validated by electromyography (EMG) recording showing significant increase in compound muscle action potential (CMAP) amplitude in hindlimb proximal (gastrocnemius) and distal plantar muscles (interosseous) of X-ray-irradiated mice at days 7, 14 and 21 post-injury, when compared with the sham-irradiated controls (FIG. 1J-K).

### LDIR Induces Hypermethylation of Fmn2 Promoter and Fmn2 Expression is Downregulated in DRGs in Response to PNI

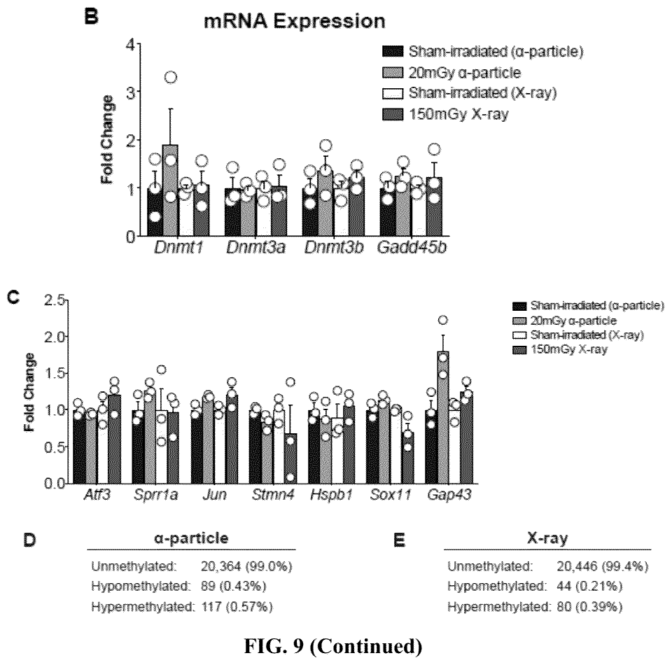

To delineate the LDIR-induced intrinsic growth program, a genome-wide CpG methylation profiling was performed by reduced representation bisulfite sequencing (RRBS) of purified DRG neurons after treating with optimal doses of α-particle (20mGy) or X-ray irradiation (150mGy). Global DNA methylation was found not to be affected by LDIR. The methylation level of CpG sites of either α-particle-or X-ray-irradiated DRG neurons was comparable to their sham-irradiated controls: ˜60% CpG sites were weakly methylated (0-10% methylated), while ˜28% CpG sites were highly methylated (>70% methylated) (FIG. 9A). The absence of global DNA methylation in LDIR-treated DRG neurons suggested no changes in the gene expression of methylation machinery such as the DNA methyltransferases (Dnmt1, Dnmt3a, Dnmt3b and Gadd45b), as confirmed by qPCR analysis (FIG. 9B). No significant differences in gene expression levels of key RAGs in DRG neurons after treated with either α-particle- or X-ray-irradiation was detected (FIG. 9C).

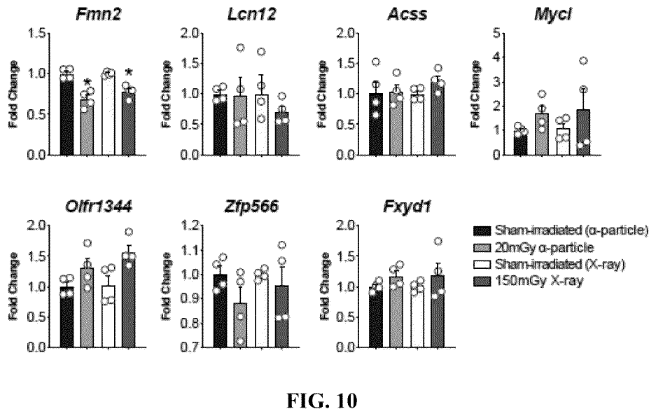

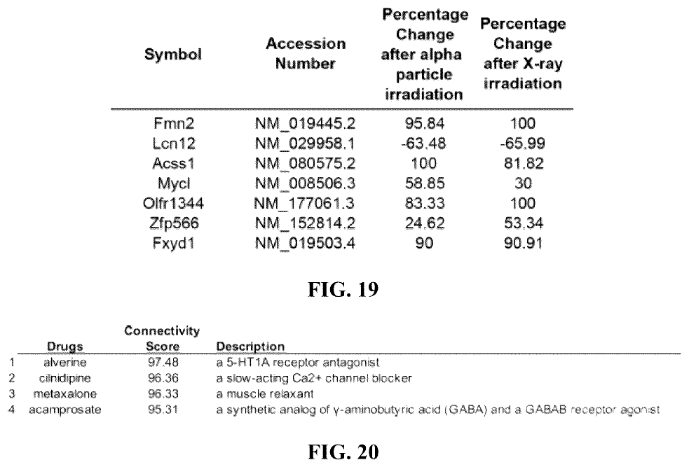

The possibility of DNA methylation at promoter regions that could be correlated with the transcriptional activation/inhibition of a corresponding gene after LDIR was then explored. 206 genes were found that differentially methylated at the CpG sites of promoter regions between α-particle-irradiated and sham-irradiated neurons (differential methylation difference >20%). Of these differentially methylated genes, 117 genes were hypermethylated and 89 genes were hypomethylated (FIG. 9D). In the X-ray-irradiated neurons, CpG island promoter of 80 genes were hypermethylated and 44 genes were hypomethylated (FIG. 9E). Of these differentially methylated genes, 7 genes were uniquely expressed in both α-particle and X-ray-irradiated DRGs that shared similar overall percentages of CpG island promoter hypermethylation or hypomethylation (FIG. 19). Correlation analysis of mRNA expression levels and DNA methylation levels of these 7 genes were performed. Notably, it was observed a significant down-regulation (hypermethylation) of Fmn2 mRNA expression in the α-particle-irradiated (32%) and X-ray-irradiated (22%) DRG neurons in cultures, when compared with sham-irradiated controls. The gene expression of the remaining 6 genes were largely unaffected regardless of their hypermethylation/hypomethylation status (FIG. 10). In fact, the actual gene expression of hypermethylated/hypomethylated genes can be unaffected or even upregulated/downregulated in some cases.

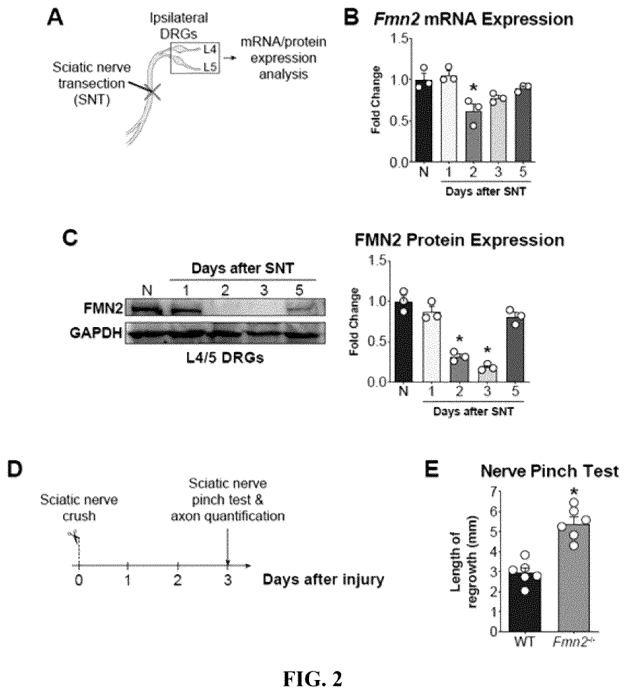

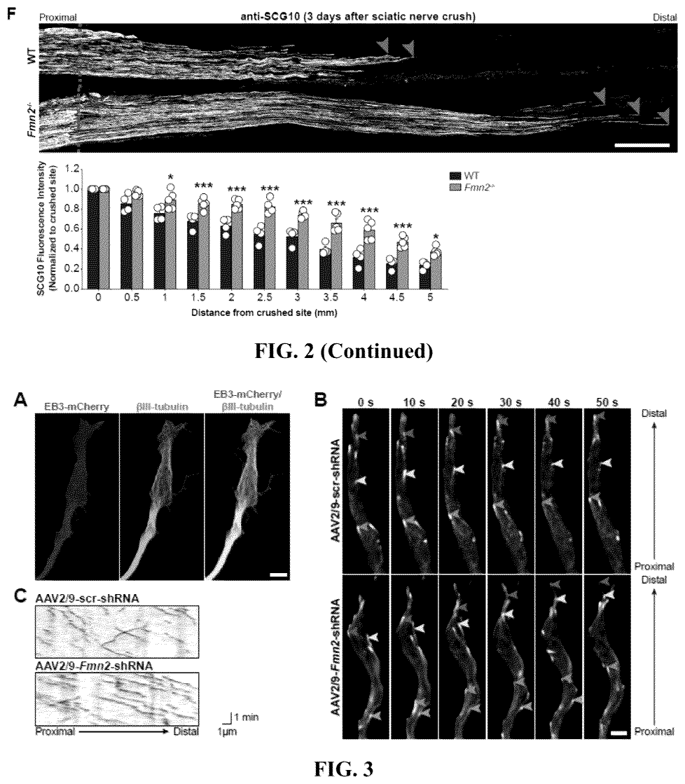

Thousands of genes are differentially expressed after PNI which orchestrate the intrinsic growth program to prime injured neurons into an actively growing state for successful axon regeneration. To verify the involvement of Fmn2 in the intrinsic growth program, mRNA and protein expression of Fmn2 was examined in injured lumbar 4 and 5 (L4/5) DRGs, which directly supply the sciatic nerve (FIG. 2A). A significant downregulation of Fmn2 mRNA (38.6%) (FIG. 2B) and protein expression (68.2-81.6%) (FIG. 2C) was detected in DRGs 2-3 days following sciatic nerve injury. These results confirm the injury-induced Fmn2 downregulation in injured DRGs and suggest Fmn2 as a potential target gene for axon regeneration.

### Genetic Ablation of Fmn2 Enhances Intrinsic Growth Capacity of Injured Neurons and Accelerates Function Recovery after PNI

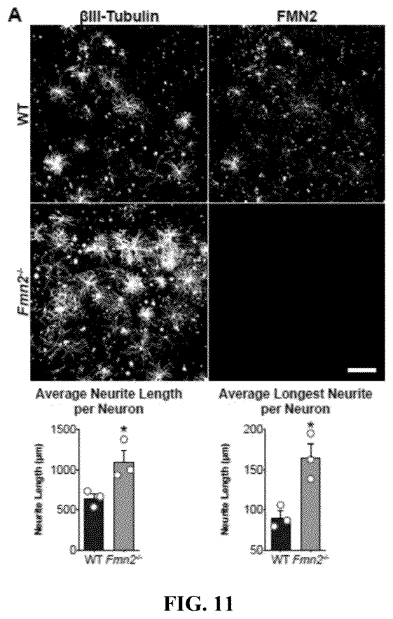

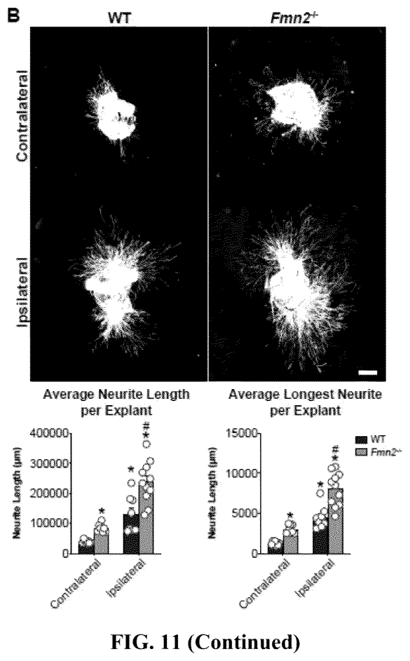

These findings were then extended to examine the intrinsic growth capacity of axotomized and preconditioned (induced maximal growth capacity) DRG neurons from a Fmn2 complete knockout (Fmn2^{-/-}) mice, which have been used extensively to study the roles of FMN2 in oocyte development. Fmn2^{-/-} mice are viable with no major defect except poor fertility in female and show no gross or histological brain differences as compared to wildtype mice. Primary DRG neurons derived from Fmn2^{-/-} mice exhibited significantly more neurite outgrowth and longer neurite length when compared with the WT mice. Total neurite outgrowth and the average longest neurite length in Fmn2^{-/-} DRG neurons were increased by 71.5% and 82.1%, respectively (FIG. 11A). It was demonstrated that a preconditioning lesion to the sciatic nerve increased neurite outgrowth of DRG explant from WT mice, while Fmn2 ablation significantly potentiated the preconditioning effect in neurite outgrowth by 81.8% (total neurite outgrowth) and 78.4% (average longest neurite) (FIG. 11B). Next, sciatic nerve crush injury on Fmn2^{-/-} mice was performed and the extent of axonal regrowth by sciatic nerve pinch test 3 days after injury was assessed (FIG. 2D). Consistent with the in vitro results, Fmn2 ablation markedly enhanced the distal extent of axonal regrowth by 83.5% (5.4±0.3 mm in Fmn2^{-/-} mice versus 2.9±0.2 mm in WT mice) (FIG. 2E). It was found that Fmn2^{-/-} mice exhibited longer SCG10-positive regenerating axons in the injured sciatic nerves compared with the WT control nerves (FIG. 2F). Significant SCG10 immunoreactivity was observed even in the far distal region of the injured nerves at 5 mm away from the injury site (FIG. 2G).

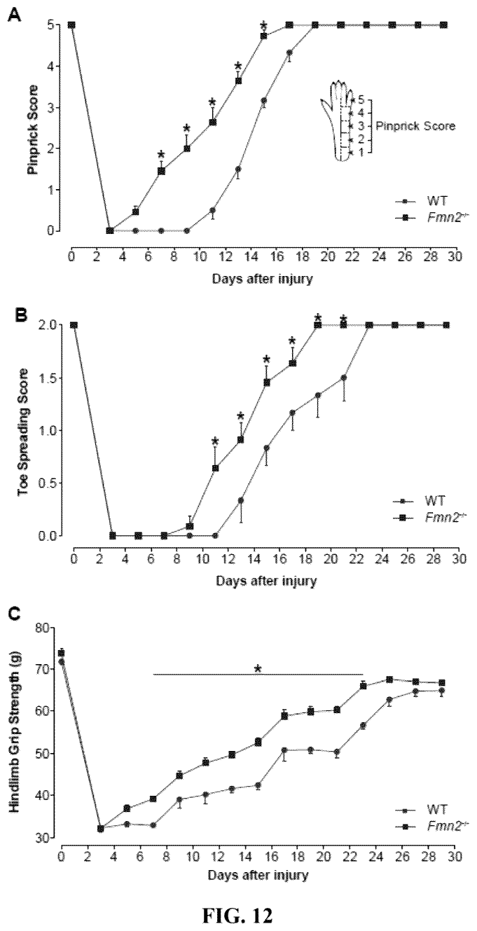

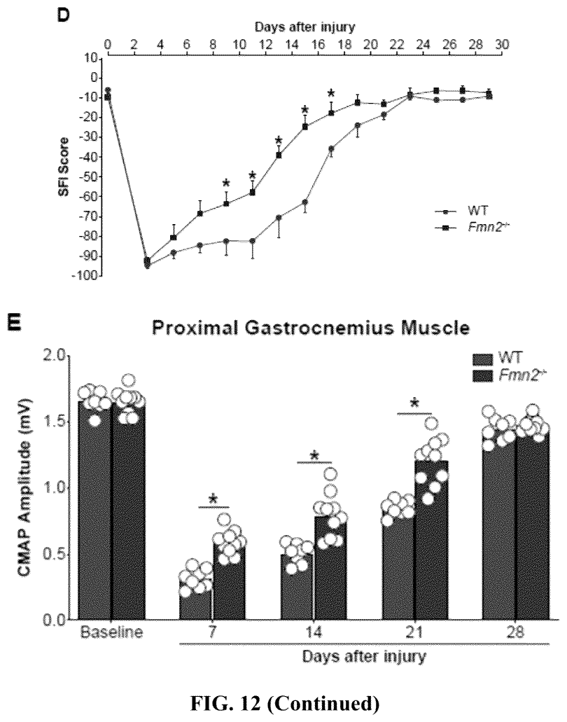

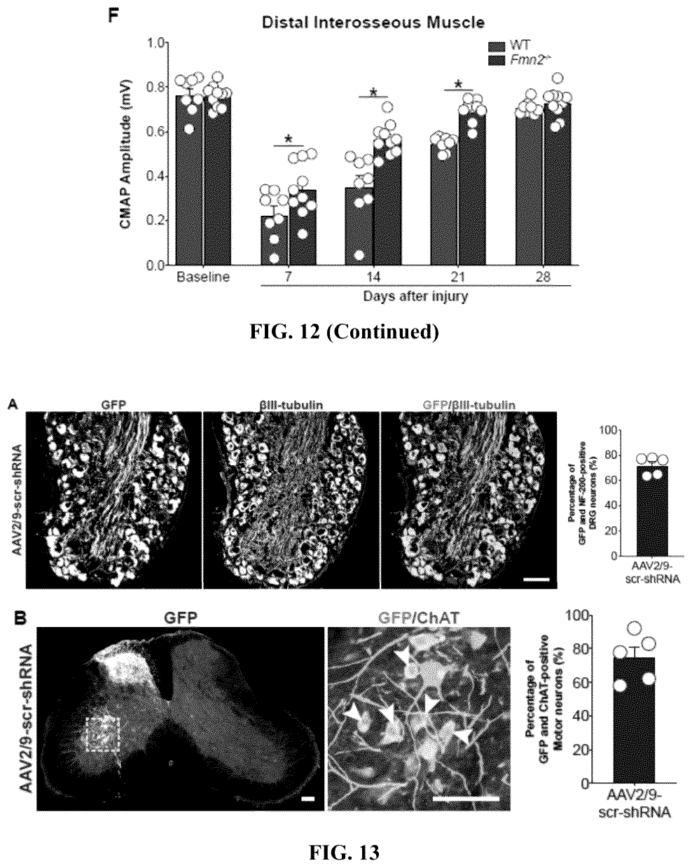

To examine if increased intrinsic growth capacity and in vivo axon regeneration in Fmn2^{-/-} mice contribute to the acceleration of function recovery after PNI, sensory and motor function recovery was assessed by an exhaustive list of animal behavior and electrophysiology tests. In Fmn2^{-/-} mice, the initial return of sensory function measured by pinprick assay was significantly earlier than the WT mice by 4 days. Fmn2^{-/-} mice regained full sensory function at day 17, which was 2 days earlier than the WT mice (FIG. 19A). For motor function recovery, Fmn2^{-/-} mice fully regained toe spreading reflexes at day 19 post-injury, 4 days earlier than the WT mice (FIG. 12B). Accelerated motor functional recovery was confirmed by grip strength tests in Fmn2^{-/-} mice, which exhibited significantly higher grip strength than controls from day 7 to 23 (FIG. 12C). Gait movement assessed by SFI showed significant motor functional recovery in Fmn2^{-/-} mice from days 9 to 17, compared with WT controls (FIG. 12D). The promoting effect observed by behavior tests was validated by muscle EMG recording showing increased CMAP amplitudes in proximal (Gastrocnemius: 1 wk 86.6%; 2 wks 56.4%; 3 wks 42.0%) (FIG. 12E) and distal plantar muscle (Interosseous: 1 wk 51.9%; 2 wks 61.6%; 3 wks 28.0%) (FIG. 12F) of Fmn2^{-/-} mice, when compared with WT controls after injury.

### Ablation of FMN2 Expression in DRGs with a High Neuronal Tropism AAV Serotype Promotes In Vivo Axon Regeneration and Function Recovery after PNI

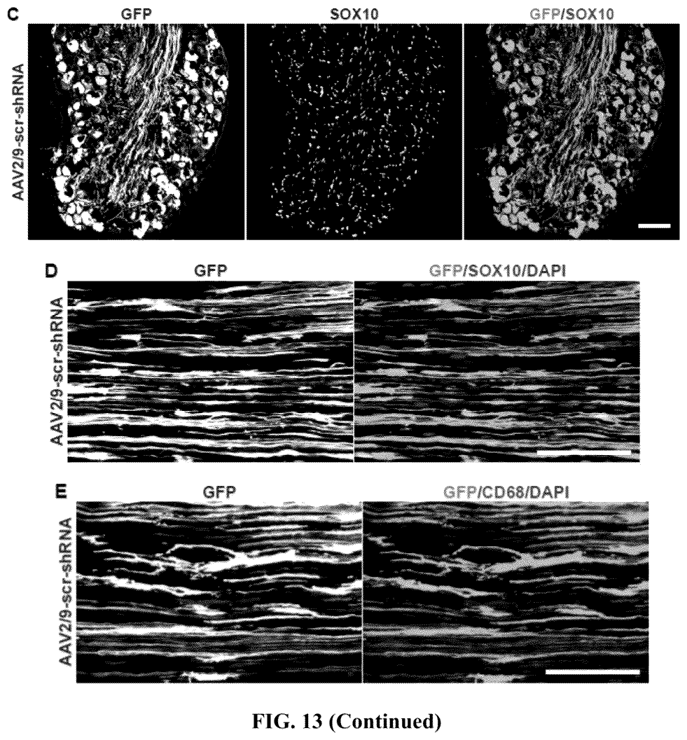

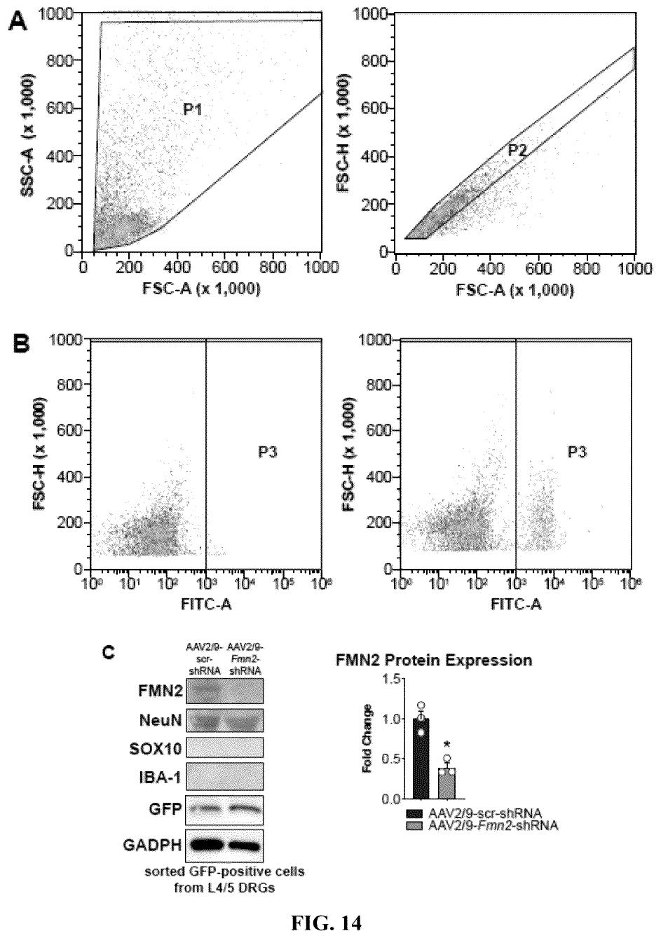

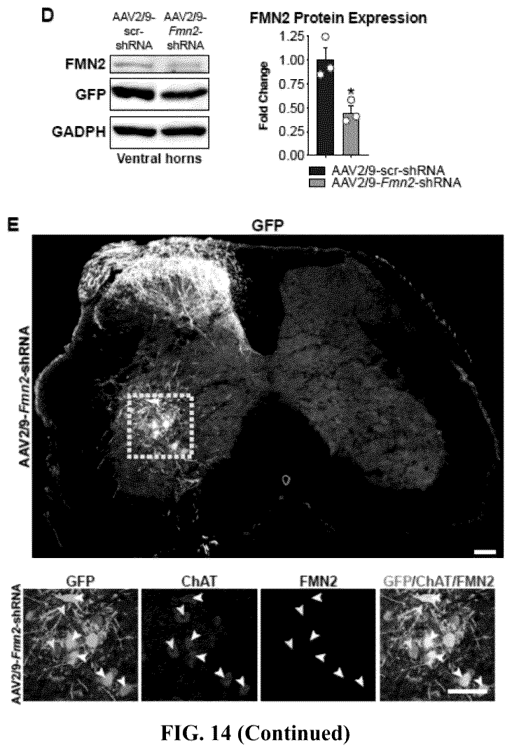

Fmn2 is almost exclusively expressed in the nervous system including the brain, spinal cord and DRG in mouse during the development; however, little is known about its mechanistic roles in axon regeneration after PNI. To overcome limitations of the global Fmn2 knockout mice, we knocked down neuronal Fmn2 expression by expressing a short hairpin RNA (shRNA) directed against Fmn2 mRNA using a recombinant adeno-associated virus (AAV2/9-Fmn2-shRNA) injected into the sciatic nerves. Among the commonly used AAV serotypes, AAV2/9 showed superior ability to transduce neurons in DRGs following peripheral routes of delivery. Mice injected with AAV2/9-scrambled (scr) shRNA-eGFP showed a high level of transduction of lumbar DRG and motor neurons at 2 weeks after sciatic nerve injection. Indeed, the eGFP signal was observed in 72% of total βIII-tubulin-positive neurons (FIG. 13A) and in 75% of ChAT-positive neurons (FIG. 13B). In contrast, eGFP signal was not found in SOX10-positive Schwann cells (FIG. 13C). To examine if AAV2/9 targeted gene delivery to non-neuronal cell populations in the sciatic nerves near to the AAV2/9 injection sites, co-localization of eGFP and SOX10/CD68 immunofluorescent signals were examined in the sciatic nerves, and it was found that virtually no eGFP immunofluorescence co-localized with SOX10-positive Schwann cells (FIG. 13D) or with CD68-positive macrophages (FIG. 13E), indicating that AAV2/9 preferentially transduces neurons but not non-neuronal cells. We further analyzed the population of transduced cells by performing flow cytometry on L4/5DRGs 2 weeks after AAV2/9 injection into the sciatic nerve (FIG. 14A-B). Our Western blot analysis demonstrated that NeuN (pan-neuronal marker), but not the glial cell makers such as SOX10 (Schwan cells) and IBA-1 (microglia), was strongly expressed in the sorted eGFP-positive cells from AAV2/9-scr-shRNA-treated or AAV2/9-Fmn2-shRNA-treated mice. In those sorted eGFP-positive neurons collected from DRGs of AAV2/9-Fmn2-shRNA-treated mice, high knockdown efficiency of Fmn2 protein expression was detected (69.2%) (FIG. 14C). Additionally, Fmn2 expression in the ventral horn of spinal cord from AAV2/9-Fmn2-shRNA-treated mice showed high knockdown efficiency (55%), when compared with AAV2/9-scr-shRNA-treated mice (FIG. 14D). To confirm the Western blot results, we performed immunohistologic studies on spinal cord to visualize the knockdown efficiency of Fmn2 in motor neurons. In AAV2/9-Fmn2-shRNA-treated mice, GFP expression was highly abundant in ChAT-positive motor neurons (white arrowheads), whereas the Fmn2 expression was greatly reduced to undetectable expression levels. In contrast, the expression of Fmn2 in untransduced ChAT-positive motor neurons remained at a high level (yellow arrowheads) (FIG. 14E). These results unequivocally demonstrate the high neuronal tropism of AAV serotype 2/9 in our studies and the specificity of Fmn2 knockdown in neurons.

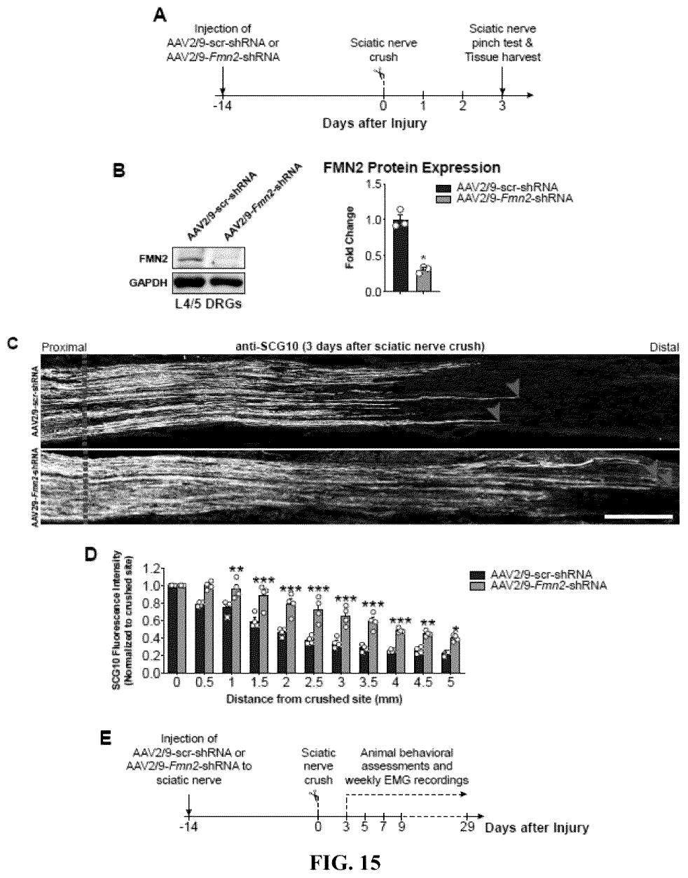

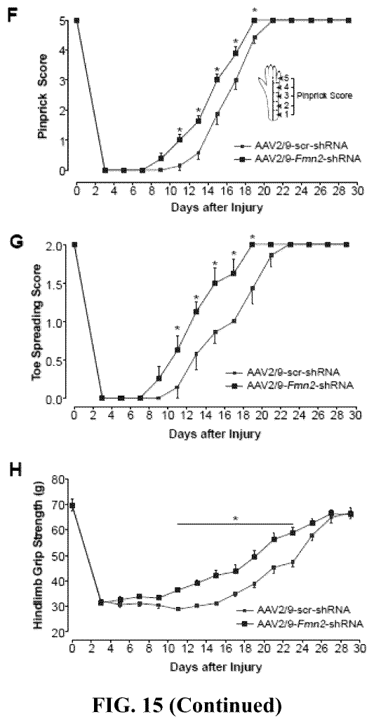

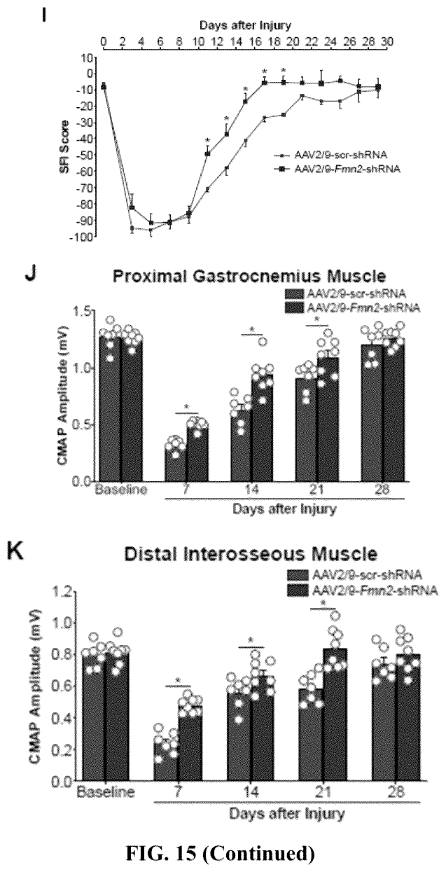

To determine the effect of neuronal Fmn2 knockdown on in vivo axon regeneration and function recovery, we injected AAV2/9-Fmn2-shRNA into the sciatic nerves (FIG. 15A). Similarly, it showed a significantly high knockdown efficiency of Fmn2 (69.2%) in L4/5 DRGs at 2 weeks after injection (FIG. 15B). We observed a significant increase in axon regeneration in AAV2/9-Fmn2-shRNA mice (5.3±0.3 mm) (FIG. 15C). The number of SCG10-positive regenerating axons 5 mm distal to the crush site was significantly higher in AAV2/9-Fmn2-shRNA mice than that in the AAV2/9-scr-shRNA control mice (FIG. 15D), suggesting that the axonal regrowth promoting effects of Fmn2 knockout are primarily mediated by neurons. As expected, our behavioural and electrophysiology studies (FIG. 15E) demonstrated that accelerated axon regeneration in AAV2/9-Fmn2-shRNA mice promoted sensory (FIG. 15F) and motor function recovery (FIG. 15G-I), and facilitated the formation of functional neuromuscular junction following sciatic nerve crush (FIG. 15J-K).

### The Mechanistical Roles of Fmn2 in Microtubule Dynamics and Post-Translational Modification of Tubulin

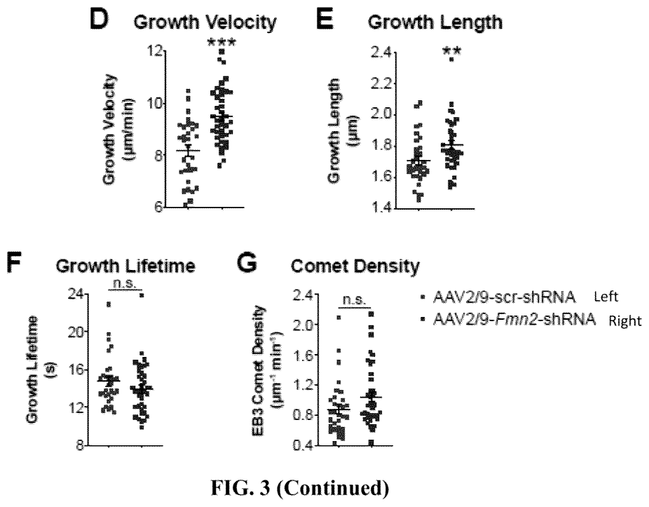

Microtubules are highly dynamic structures which undergo continuous assembly (polymerization) and disassembly (depolymerization). The dynamic growing plus-end of microtubules act as “sensors” of cellular microenvironment, which allow the rapid reorganization of the cytoskeleton for successful axon regeneration. Microtubule dynamics can also be affected by the post-translational modifications such as tubulin acetylation and deacetylation. Therefore, microtubule dynamics were first assayed quantitatively using high-resolution time-lapse confocal microscopy and fluorescently tagged microtubule end-binding protein 3 (EB3) in peripheral axons. To this end, DRG neurons from adult mice were purified and transduced the DRG neurons with AAV2/9-EB3-mCherry. Four days upon transduction, the growth cones were categorized as both GFP-positive and EB3-positive during the live cell imaging with the confirmation of βIII-tubulin immunoreactivity after fixation (FIG. 3A) and a series of 5-min time-lapsing images (with a time interval of 2 s per frame) were captured. Microtubule dynamics were quantified by tracking the displacement of EB3 comets (and therefore growing microtubules) at the microtubules plus-ends in sequential frames of time-lapsing images. (FIG. 3B) (Movie S1). Kymographs analysis of EB3-mCherry signals exhibited EB3-mCherry comet trajectories with increasing comet speed at the distal ends of Fmn2-deficient DRG neurons (FIG. 3C). The movement of these EB3 comets can be used to determine microtubule dynamics quantitatively by using an automated MATLAB-based software plusTipTracker. Compared with AAV2/9-scr-shRNA control DRG neurons, the speed of microtubule polymerization (growth velocity) was markedly increased by 16.1% in Fmn2-deficient DRG neurons, indicating that the Fmn2-deficient microtubules grew significantly faster than the control microtubules (8.20±0.21 μm/min vs. 9.52±0.17 μm/min). Fmn2-deficient DRG neurons also displayed a significant increase in microtubule growth length, which represented the total displacement of EB3 comets before they were paused or catastrophe (1.71±0.03 μm in AAV2/9-scr-shRNA neurons versus 1.81±0.03 μm in AAV2/9-Fmn2-shRNA neurons) (FIG. 3E). Fmn2 knockdown did not affect the duration of microtubule polymerization (growth lifetime) (FIG. 3F) and density of EB3 comets in the growth cones (FIG. 3G). Our data suggests that Fmn2 knockdown increases the microtubule dynamics in growth cones, which is consistent with previous studies on the role of Fmn2 as a regulator of growth cone dynamics in cultured chick spinal commissural neurons and zebrafish.

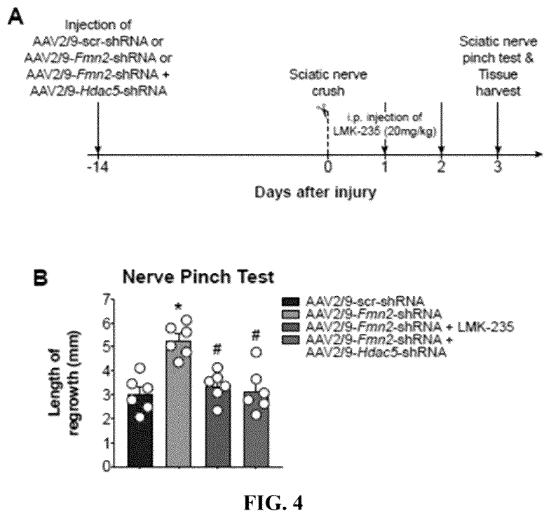

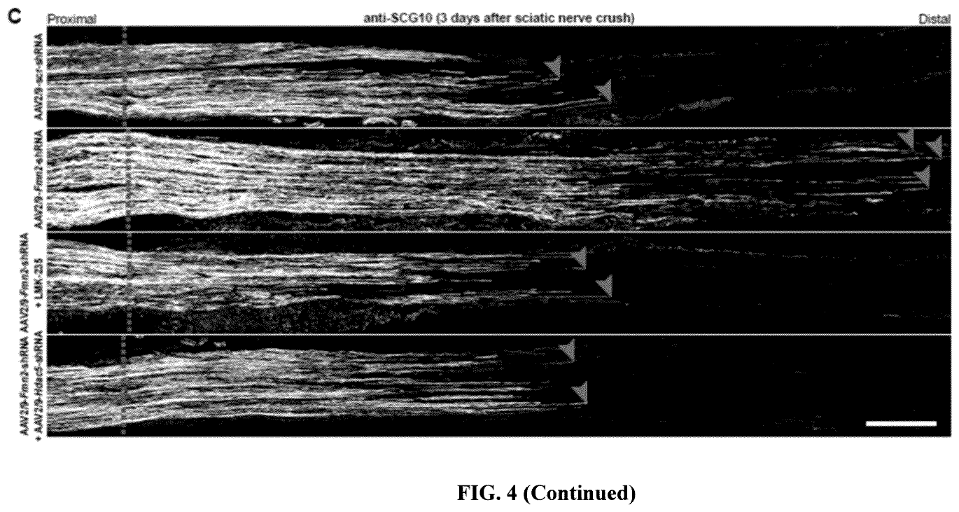

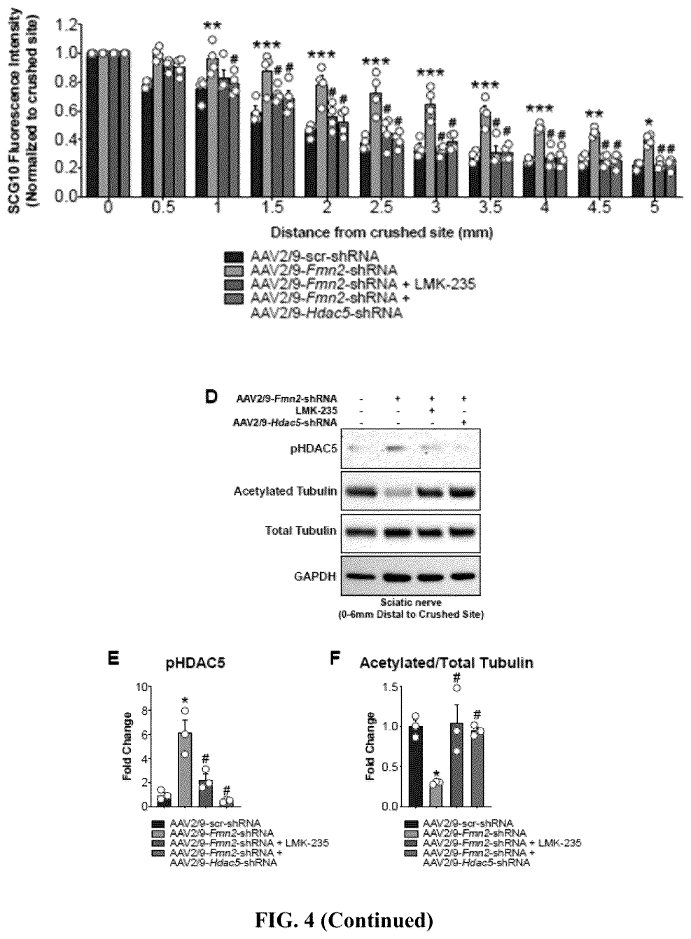

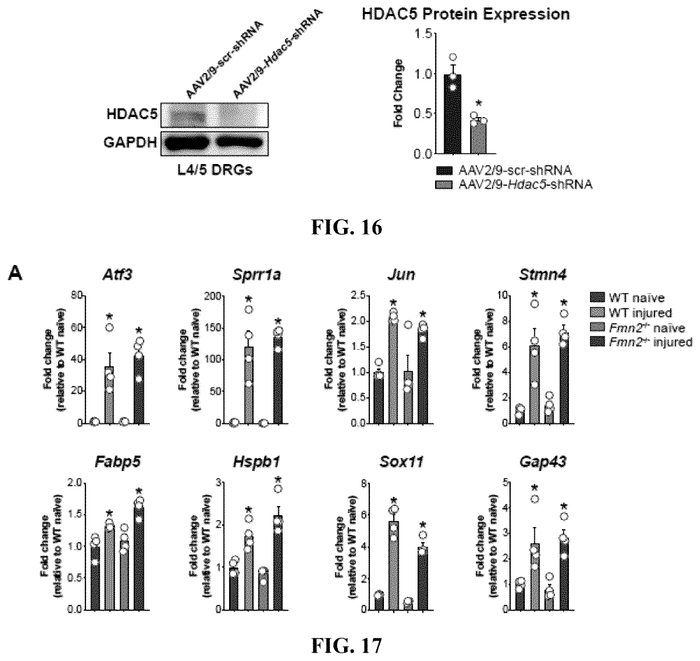

Formin protein including Fmn2 contains highly conserved formin homology 1 (FH1) and FH2 domains at the C-terminal. A growing body of work suggests that FH1 and FH2domains modulate microtubule stability and dynamic by inducing α-tubulin acetylation. Histone deacetylase 5 phosphorylation (HDAC5)-medicated α-tubulin deacetylation is crucial for axon regeneration. For instances, sciatic nerve injury induces phosphorylation of HDAC5in injured axons in vivo and it has been shown that pHDAC5 catalyses deacetylation of α-tubulin in axons to promote axon regeneration. To examine the mechanistic roles of neuronal Fmn2 expression in axon regeneration, and more specifically, whether HDAC5 is responsible for the Fmn2-deletion induced growth-promoting effect, LMK-235 were injected intraperitoneally, a selective inhibitor of HDAC4 and HDAC5 which has been reported to preferentially inhibit HDAC5 activity at low-dose exposure immediately following sciatic nerve crush and assessed in vivo axon regeneration by nerve pinch test (FIG. 4A). At 3 days after sciatic nerve injury, LMK-235 completely eliminated the axon regrowth-promoting effect in AAV2/9-Fmn2-shRNA mice to a level comparable to that of AAV2/9-scr-shRNA control mice. To rule out the possibility of any non-specific inhibition of HDAC4 even at low-concentration of LMK-235, HDAC5 expression was knocked down by expressing a shRNA directed against Hdac5 mRNA using AAV2/9 (AAV2/9-Hdac5-shRNA) injected into the sciatic nerves resulting in a similar reduction in the rate of distal axon regeneration and number of SCG10-positive regenerating axons at 3 days after injury (FIGS. 4B-D). Consistent with observations in AAV2/9-Fmn2-shRNA knockdown experiments, AAV2/9-Hdac5-shRNA also significantly reduced HDAC5 protein expression in L4/5 DRGs by 57.8% (FIG. 16). Next, pHDAC5 and acetylated tubulin protein expression was examined in 0-6 mm sciatic nerve segments distal to the crush injury site in the AAV2/9-scr-shRNA and AAV2/9-Fmn2-shRNA mice at 3 days after the injury by Western blot analysis (FIG. 4E). In the 0-6 mm sciatic nerve segments distal to the crush site where active axon regeneration is taking place in the AAV2/9-Fmn2-shRNA mice according to our nerve pinch test results in FIG. 4B, densitometry of the resulting bands indicated a 5.13-fold increase in pHDAC5 protein at day 3 after injury (FIG. 4F) which appeared to correlate with the decreased acetylated tubulin expression (70.3%) in AAV2/9-Fmn2-shRNA mice (FIG. 4G). Similarly, either pharmaceutical blockade of HDAC5 by LMK-235 (70%) or gene silencing of HDAC5 using AAV2/9-Hdac5-shRNA (77%) substantially reduced the protein expression of pHDAC5, leading to increased acetylated tubulin expression in sciatic nerves of AAV2/9-Fmn2-shRNA mice at 3 days after injury (FIGS. 4E-G).

### Small Molecule Bioinformatics Analysis of Fmn2^{-/-} Gene Expression Signature and the Identification of a Therapeutic Small Molecule Metaxalone

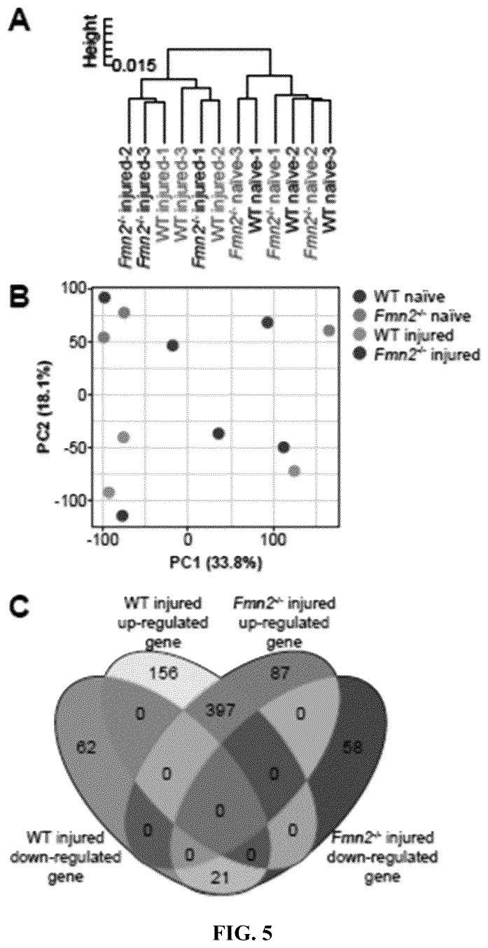

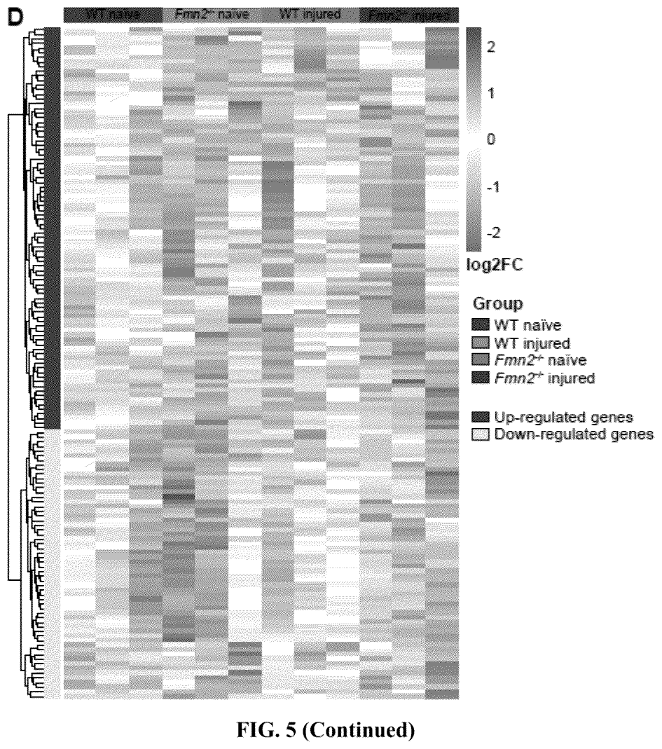

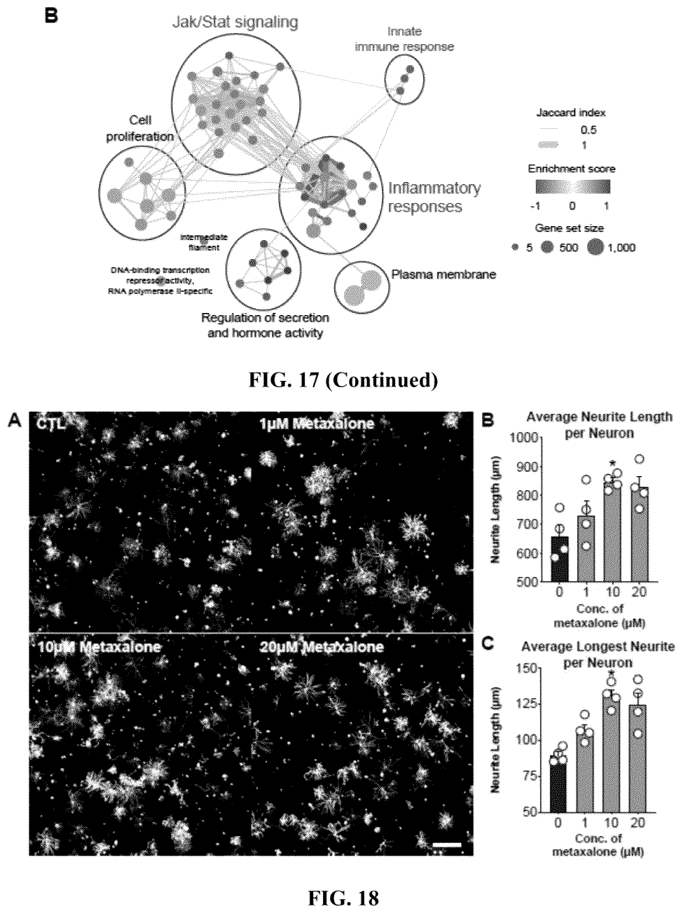

Injury of axons in the PNS induces intrinsic growth capacity of neurons and transcriptional changes in gene expression that promotes axon regeneration. It was therefore reasoned that bioactive small molecules associated with the gene regulatory network of Fmn2deletion to switch on the intrinsic growth capacity of injured neurons results in accelerated in vivo axon were identified they could be used for regeneration and recovery of function. To achieve this, a whole transcriptome analysis was performed and small molecule bioinformatics analysis on L4/5 DRGs from Fmn2^{-/-} and their aged-matched WT mice 5 days after PNI. Hierarchical clustering and principal component analysis showed a clear separation between contralateral and ipsilateral DRGs regardless of genotypic differences (FIG. 5A-B). After injury, there were 563 and 636 genes differentially expressed (DE) in Fmn2^{-/-} and WT DRGs, respectively. Of these DE genes, 397 genes were up-regulated, and 21 genes were down-regulated in both Fmn2^{-/-} and WT injured DRGs (FIG. 5C). Specifically, key RAGs such as Atf3, Sprr1a, Jun, Stmn4, Fabp5, Hspb1, Sox11 and Gap43 were highly upregulated to a similar extent in both Fmn2^{-/-} and WT injured DRGs. Fmn2-deletion did not affect the RAG expression after PNI (FIG. 17A). A total of 145 genes (87 up-regulated and 58 down-regulated) were uniquely DE only in Fmn2^{-/-} mice, but not in the WT mice after injury (FIG. 5D). Gene set enrichment analysis (GSEA) was performed to identify core gene regulatory network governing the enhanced regenerative capacity in Fmn2^{-/-} mice. A significant enrichment in signalling pathways associated with inflammatory responses such as Jak/Stat and chemokine/cytokine-related signalling pathways was observed (FIG. 17B).

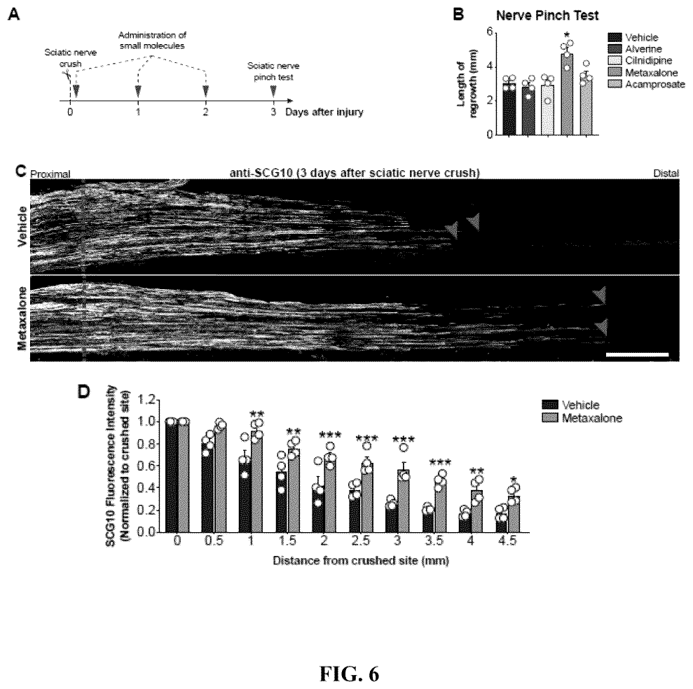

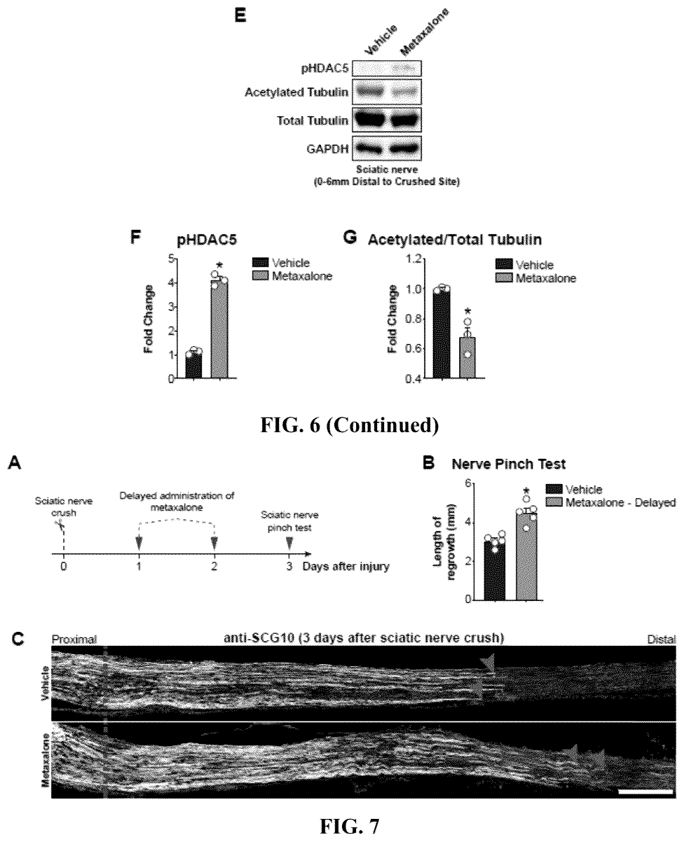

Next, these 145 DE genes as gene expression signatures were utilized to query a public database Library of Integrated Network-Based Cellular Signatures (LINCS), and to match with gene expression profile derived from cell lines treated with small molecules based on connectivity and specificity scores (see Methods for details). Four small molecules were then selected with connectivity score over 95 (score 100 being the highest) for experimental validation (FIG. 20) using sciatic nerve pinch test (FIG. 6A). FDA-approved small molecule metaxalone showed a marked increase of distal sensory axonal regrowth (4.82±0.32 mm in metaxalone-treated mice versus 3.05±0.16 mm in vehicle controls) 3 days after sciatic nerve crush injury (FIG. 6B). In line with the pinch test results, a significant increase in the number of SCG10-positive regenerating axons in metaxalone-treated mice at 4.5 mm distal to the crush injury site were observed when compared with vehicle controls (FIG. 6C-D). As expected, metaxalone induced a substantial increase in protein expression of pHDAC5 (FIG. 6E-F) associated with the reduction of tubulin acetylation (FIG. 6G) in regenerating axons of sciatic nerves 3 days post-injury, demonstrating the recapitulation of growth-promoting mechanism induced by Fmn2-deletion. To evaluate the direct effect of metaxalone on purified DRG neurons, axotomized DRG neurons were treated with increasing dose of metaxalone and neurite outgrowth was increased almost linearly until reaching a plateau at 20 μM (FIG. 18). Our results demonstrated that metaxalone could enhance the intrinsic growth capacity of injured DRG neurons directly.

### Delayed Metaxalone Treatment Promotes Axon Regeneration and Function Recovery

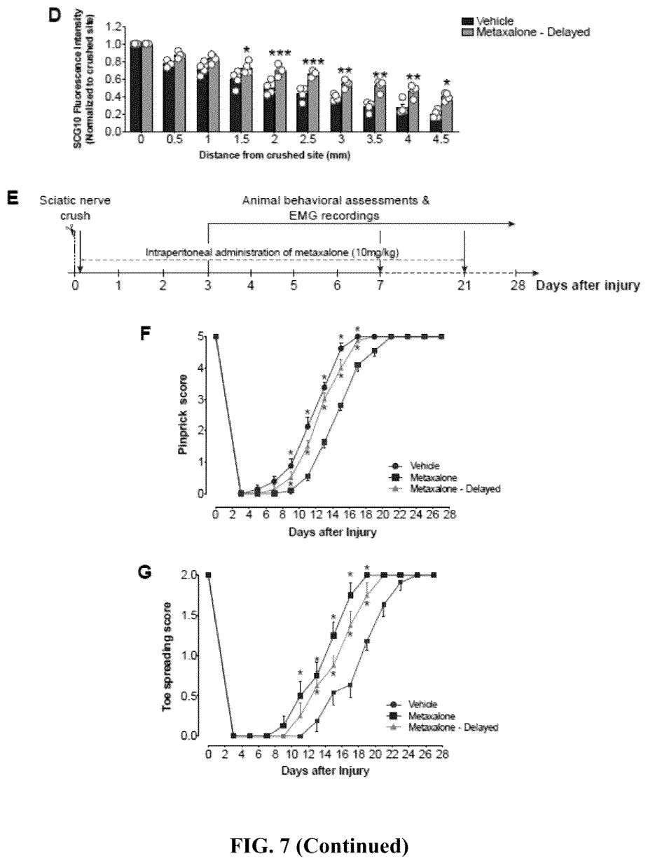

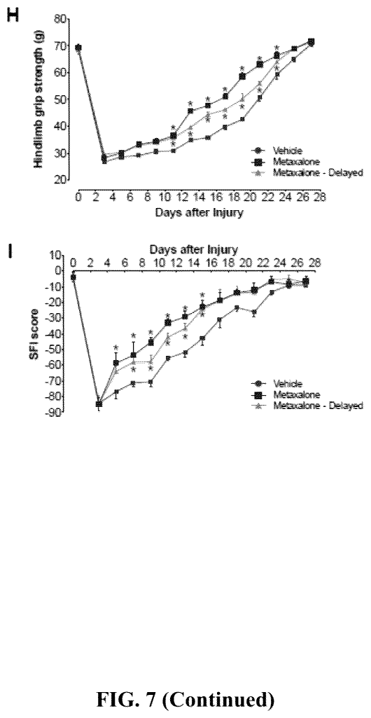

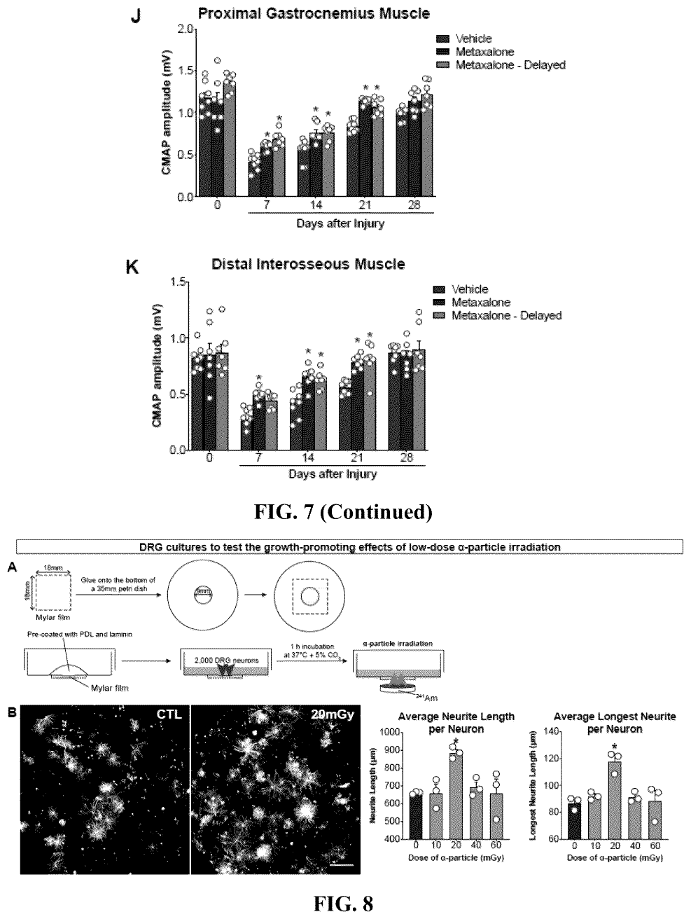

To simulate a clinically relevant situation where hours could elapse before any treatment is possibly available, the efficacy of post-injury treatment with metaxalone delayed by 24 hours was first evaluated in promoting in vivo axon regeneration. Metaxalone was administrated one day after sciatic nerve crush injury. At 3 days post-injury, a nerve pinch test was performed to assess the extent of axonal regrowth (FIG. 7A). Similar to the mice treated with metaxalone immediately after injury (4.82±0.32 mm) (see FIG. 6B), accelerated axon regeneration was observed in the mice with delayed metaxalone treatment by 24 hours (4.50±0.25 mm) (FIG. 7B). By examining the expression of SCG10, it was found that the regeneration index at the region over 4.5 mm was similar in metaxalone-treated (0.34) (see FIG. 6D) and delayed-metaxalone-treated mice (0.40) (FIG. 7C-D), indicating that metaxalone treatment delayed even by 24 hours did not affect both the length and the number of regenerating axons past the crush site.

Finally, to further validate the therapeutic applicability of metaxalone in function recovery when treatment initiation was delayed until 24 hours after PNI. Adult mice received 10 mg/kg of metaxalone intraperitoneally once a day immediately after PNI for 21 consecutive days or delaying daily metaxalone treatment to 24 hours post-injury for 20 consecutive days (FIG. 7E). Metaxalone-treated and delayed-metaxalone-treated mice showed initial response to pinprick stimuli (reaching score 1) as soon as on day 9 and day 10 post-injury, respectively. Mice regained full sensory pinprick response 4 days (metaxalone-treated) and 2 days (delayed-metaxalone-treated) earlier than vehicle controls (FIG. 7F). For motor function recovery, metaxalone-treated mice took 14 days to recover 50% of toe spreading reflex (reaching score 1) and 19 days to fully regain toe spreading reflex. Delayed-metaxalone-treated took 1 more day than metaxalone-treated mice to recover 50% of toe spreading reflex as well as to fully regain toe spreading reflex. The vehicle-treated mice were only able to recover 50% of toe spreading reflex in 18 days and required 7 more days to fully recover (FIG. 7G). Metaxalone-treated and delayed-metaxalone-treated mice exhibited significantly higher hindlimb grip strength recovery than controls from day 11 to 23 (FIG. 7H). Metaxalone and delayed metaxalone treatment displayed the most rapid SFI improvement over the course of recovery after injury compared to vehicle alone. At 15 days post-injury (halfway through the recovery period), the average SFI was increased substantially in mice treated with metaxalone immediately (73.0%) and delayed metaxalone treatment by 24 hours (71.4%), while the average SFI of vehicle-treated mice was increased by 49.3% when compared with the SFI values at day 3 post-injury (FIG. 7I).

Consistent with the animal behavioral data, EMG recordings of metaxalone-treated and delayed-metaxalone-treated mice in the proximal gastrocnemius revealed significant improvement in muscle activities at week 1 to 3 post-injury, when compared with the vehicle controls. The increase in muscle activity of metaxalone-treated mice reached significance levels in the most distal interosseous muscles during the first 3 weeks post-injury, while a significant increase in muscle activity was detected in delayed-metaxalone-treated mice at week 2-3 post-injury compared to vehicle controls (FIG. 7J-K). In conclusion, our bioinformatic analysis successfully identified an FDA-approved small molecule metaxalone not only recapitulated the growth-promoting effects of Fmn2 ablation in peripheral nerve repair, but also able to delay intervention by such a large therapeutic time window is advantageous in the translation of findings into clinical application.

This work reveals that neuronal Fmn2-deletion promotes axon regeneration and function recovery by increasing microtubule dynamics and post-translational modification such as tubulin deacetylation at the distal ends of growing axons. Fmn2 has been shown to bind and interact with microtubule, and to induce α-tubulin acetylation. Tubulin deacetylation, which is induced by HDAC, produces highly dynamic microtubules and plays a key role in axon regeneration. However, little is known about the mechanisms underpinning Fmn2 function in peripheral nerve regeneration. In this study, it was first examined the potential beneficial effects of LDIR on in vitro and in vivo axonal regrowth in axotomized DRG neurons. It was hypothesized that the alternation in gene expression profiling following LDIR contributes to the beneficial adaptive responses after PNI. However, our RRBS and transcriptional analysis showed that LDIR did not affect the global DNA methylation and key RAG expression in axotomized DRG neurons. There is a lack of consistent correlation between the DNA methylation and RAG expression. For instance, either pharmacological induction or suppression of global DNA methylation reduces neurite outgrowth in axotomized DRG neurons by suppressing gene expression of a numbers of key RAGs including Arg1, Sprr1a, Gadd45a, and Gap43. An in-depth bioinformatics analysis was performed, which revealed a previously unrecognized functional role of Fmn2, with over 96% of DNA hypermethylation occurring in its promoter region (downregulation of Fmn2 expression), in axon regeneration. Our RNA-seq analysis showed that ablation of Fmn2 did not alter RAG expression in injured DRGs, but interestingly, Fmn2-deletion induced activation of transcriptional networks regulating immune responses such as the Jak-Stat signalling pathway, and pathways involved in chemokine and cytokine production were uniquely enriched only in injured Fmn2^{-/-} DRGs. It is well documented that pSTAT3, acts as a transcription factor mediating virtually all cytokine-driven signalling, is a critical transforming factor of the neuronal pro-regenerative state. Our GSEA analysis suggests that Fmn2-deletion might create a favourable anti-inflammatory microenvironment for peripheral nerve repair. It was then confirmed that the injury-induced Fmn2 downregulation in DRGs at the early time points after injury whereas robust axon regeneration occurs. Using a combination of approaches, the relationship between the Fmn2-deletion and microtubule dynamics, and the acceleration of axon regeneration and function recovery was elucidated.

Microtubule plus-end polymerization is required for regenerating axons through cycles of polymerization and depolymerization (dynamic instability), which allows microtubules at the growing ends to probe the intracellular environment and to facilitate the projection of regenerating axons back to their original target areas (i.e., skin, muscle). Microtubule dynamic instability is therefore critical for growth cone guidance, guiding the regenerating axons along its correct path. In the current study, it was shown that Fmn2-deletion prolongs the high microtubule dynamic phase at the microtubule plus-ends of growing axons by increasing the speed of microtubule polymerization (growth velocity) and duration of microtubule polymerization (growth length). It was also hypothesized that following microtubule polymerization; post-translational modifications would have a direct effect on microtubule dynamics via tubulin acetylation. The data herein shows that pHDAC5 was highly upregulated exclusively at the regenerating axons of AAV2/9-Fmn2-shRNA mice 3 days after sciatic nerve crush. Inhibition of HDAC5 activity through either LMK-235 or gene silencing of Hdac5 using AAV2/9-Hdac5-shRNA, both treatments eliminated the axon growth-promoting effect of Fmn2-deletion completely in AAV2/9-Fmn2-shRNA mice (FIG. 4B-C). These results agree well with the observation that peripheral axonal injury induced nuclear export of HDAC5 from the nucleus to the cytoplasm and subsequent HDAC5 anterograde transport into the axons. Additionally, HDAC5, but not HDAC1-4 and 6, is identified as a novel injury-dependent tubulin deacetylase catalyzing the deacetylation of α-tubulin in axons. By inhibiting the expression of HDAC6 and pharmacologically blocking HDAC6 activity improve neurite outgrowth in cortical neurons on inhibitory substrates of myelin such as myelin-associated glycoprotein and chondroitin sulfate proteoglycan. However, the promoting effect of HDAC6 in permissive PNS microenvironment is negligible in which HDAC6 knockdown has no significant effect on axon regeneration. Unlike HDAC6, HDAC5 knockdown in DRG neurons does not affect the basal level of acetylated tubulin indicating that HDAC5 is not involved in regulating tubulin acetylation under basal conditions but only in response to injury. Whereas sciatic nerve injury induces phosphorylation (activation) of HDAC5 in injured axons in vivo, relatively low pHDAC5 expression was detected in the uninjured axons. Recent reports also highlight the fact that increases tubulin polymerization and microtubule dynamics show promising results for axon regeneration and function recovery after injury. For instance, genetic ablation of TUBB3 markedly elevated the level of acetylated tubulin (stable form) and impaired microtubule polymerization at the growth cones of cultured DRG neurons. The reduced microtubule dynamics impaired neurite outgrowth of TUBB3-deficient neurons, resulting in delayed sensory function recovery after PNI. Epothilone B induced rapid tubulin polymerization at the growing tips of cultured rat cortical neurons and promoted in vivo axon regeneration after spinal cord injury. Similarly, paclitaxel induced active tubulin polymerization at the growth cones and facilitated in vivo axon regeneration, and partial function recovery in rats with spinal cord injury. Nevertheless, chronic exposure to epothilone B or paclitaxel is known to cause chemotherapy-induced peripheral neuropathy when used at high doses in animals. It is also notable that formin protein has been known as an actin nucleator and plays a key role in formin-dependent actin nucleation, which is the first step in actin polymerization. Understanding how the changes in actin cytoskeleton in the growth cone of Fmn2-deficient neurons and the involvement of Fmn2 in actin-microtubule crosstalk would be an important area for further research on axon regeneration.

In the final proof-of-concept experiment, a small molecule bioinformatics was performed analysis using the gene expression profile signature of Fmn2^{-/-} to query a public database LINCS, which consists of 44,328 bioactive small molecules and 1,234 of them are FDA-approved small molecules. Metaxalone, an FDA-approved drug, was identified, which has been used as a skeletal muscle relaxant to treat a broad range of skeletal muscle disorders associated with acute and painful musculoskeletal conditions (i.e., diabetic neuropathy) for over 2 decades. Additionally, it was demonstrated that metaxalone treatment delayed by 8 hours post-injury still can accelerate substantial axon regeneration and function recovery to a level comparable to that of metaxalone treatment immediately after injury, which provides a highly relevant treatment strategy for clinical setting.

Taken together, the current study provides novel insights into the development of an FDA-approved small-molecule based therapy for treating PNIs. It usually takes over 15 years from identification of bioactive compound to FDA approval for clinical application. Repurposing of existing FDA-approved drug such as metaxalone would be time-saving that normally Phase I clinical trial can be skipped. This is an important proof-of-concept study to demonstrate that FDA-approved small molecules identified by bioinformatics analysis can be readily used as a potential therapy not only for PNIs, which can also be applied to transitional drug discovery across a range of different nervous system disorders.

### STAR Methods

### Resource Availability

### Materials Availability

All unique reagents generated in this study are available from the lead contact upon reasonable request.

### Animals

Animal experiments were performed in accordance with the experimental procedures approved by the Animal Research Ethics Sub-Committee at the City University of Hong Kong. In compliance with the American Veterinary Medical Association (AVMA) Guidelines on euthanasia, carbon dioxide asphyxiation was performed for animal euthanasia. Adult male C57BL/6 or Fmn2 knockout (Fmn2^{-/-}) mice (8-12 weeks old) were used in both in vitro and in vivo experiments. The mice were provided with food and water ad libitum, with a 12:12 h light-dark cycle.

Fmn2^{-/-} mice with 129S6/SvEvTac background were obtained from Jackson laboratory. To delete Fmn2, a 1,300-base pair (bp) sequence for the proline-rich FH1 domain of Fmn2 gene was replaced by a 1,257-bp neomycin-resistance gene followed by a stop codon to terminate transcription of Fmn2. Fmn2^{-/-} mice were viable, and no morphological and functional deficits were observed in the nervous system. Male Fmn2^{-/-} mice were fertile and decreased fertility was found in female Fmn2^{-/-} mice. Therefore, only male Fmn2^{-/-} mice were used in current study. PCR genotyping was performed with genomic DNA isolated from the tail. Forward 5′-CTAATTGTGGCTGCCCTTGT-3′ (SEQ ID NO:13) and reverse 5′-AGGTGGCAATGTCAGGATTC-3′ (SEQ ID NO:14) primers were used for PCR amplification of wild-type Fmn2 with product size of 318 bp. Forward 5′-CTTGGGTGGAGAGGCTATTC-3′ (SEQ ID NO:15) and reverse 5′-AGGTGAGATGACAGGAGATC-3′ (SEQ ID NO:16) primers were used for PCR amplification of mutant Fmn2 with product size of 280 bp.

### Surgery

Sciatic nerve crush or transection injury was performed in anesthetized adult male C57BL/6 or Fmn2^{-/-} mice (8-12 weeks old) as previously described. Briefly, the left sciatic nerve was exposed at mid-thigh level. For crush injury, the exposed nerve was crushed with smooth forceps (Dumont #5/45 Forceps, Fine Science Tools) at the sciatic notch for 15 seconds. For sciatic nerve transection, an epineural suture was first made at the level of external rotator muscle, just distal to the sciatic notch. The exposed nerve was then transected using a pair of ophthalmic microscissors just distal to the suture. After injury, the overlying muscle and skin were sutured using a 5-0 suture (Ethilon), and the mice were allowed to recover on a heated pad, and returned to their home cages after recovery. The surgeon was blinded to the genotypes and treatments.

### Whole-Body X-Ray Irradiation

Adult male C57BL/6 mice (8-12 weeks old) were irradiated by a cabinet irradiator X-Rad 320 (Precision X-Ray) at 200 kV and 10 mA equipped with a beam conditioning filter (1.5 mm Aluminum, 0.25 mm Copper and 0.75 mm Tin) at a focus-to-surface distance of 70 cm. To ensure that each mouse inside the pie cage received the same amount of radiation, dosimetry inside the pie cage was measured by a dosimeter before experiment. During irradiation, pie cage was placed on a slowly rotating turntable to allow uniform distribution of X-ray irradiation. Mice received a single exposure to X-ray irradiation at different doses (1-5 Gy). Sham irradiated control mice were placed inside the irradiator without switching on the X-ray. Ex vivo DRG explant cultures were performed 5 days after the whole-body X-ray irradiation (see FIG. 8F for detailed experimental paradigm). For sciatic nerve pinch test and function recovery assessment after PNI, whole-body X-ray irradiation was performed on the injured mice immediately after sciatic nerve crush injury (see FIGS. 1A and D for detailed experimental paradigm).

### Ex Vivo DRG Explant Culture

Ex vivo DRG explant cultures were prepared from adult (8-12 weeks old) male C57BL/6, Fmn2^{-/-} mice and their age-matched WT littermates as described. Briefly, DRGs were dissected out, cleaned of spinal and peripheral roots and plate onto 8-well chambers (Millipore) coated with poly-D-lysine (100 μg/ml) (Sigma-Aldrich) and a thin layer of Matrigel (BD Biosciences). The DRG explants were then incubated in full Neurobasal (NB) medium supplemented with B27, 200 mM L-glutamine, penicillin/streptomycin, 50 ng/ml NGF (Gibco), 2 ng/ml GDNF and 10 μM Ara-C (Sigma-Aldrich). After 48 hours, DRG explant cultures were fixed with 4% paraformaldehyde (PFA), and immunostained with anti-βIII-tubulin antibody for neurite outgrowth assay.

### Primary Dissociated DRG Culture

Primary cultures of dissociated DRG neurons were prepared from adult male C57BL/6 or Fmn2^{-/-} mice (8-12 weeks old) and their age-matched WT littermates as described. Briefly, DRGs were dissected out from the mice and mildly digested in collagenase/dispase II (Roche Diagnostics) solution, trypsinized and mechanically dissociated using flame polished Pasteur pipettes with three different diameters. DRG neurons were incubated in full NB medium.

For extraction of genomic DNA or total RNA from cultured DRG neurons, DRG neurons were plated on a thin layer of Mylar film (for alpha particle irradiation) or glass bottom dishes (for X-ray irradiation) pre-coated with poly-D-lysine (100 μg/ml) and laminin (10 μg/ml) (Sigma-Aldrich), at a fixed cell density of 3,000 cells/cm². The DRG neurons were cultured in supplemented Neurobasal (NB) medium with B27, 200 mM L-glutamine, penicillin/streptomycin, 50 ng/ml NGF (Gibco), 2 ng/ml GDNF and 10 μM Ara-C (Sigma-Aldrich) for 17 hours.

### Alpha-Particle and X-Ray Irradiation on Cultured DRG Neurons

For α-particle irradiation, a planar ²⁴¹Am with α-particle energy of 5.49 MeV under vacuum and activity of 4.26 kBq was placed under a 9 mm-hole covered by Mylar film. DRG neurons were exposed to α-particle irradiation for 1 minute, which corresponded to the absorbed dose of 20mGy. Sham irradiation was performed by exposing the neurons to a non-radioactive source (i.e. plastic film) for 1 minute.

For X-ray irradiation, the glass bottom dish containing the DRG neurons were placed at the centre of the X-ray irradiator and exposed to a single dose of 150mGy, using a cabinet irradiator X-Rad 320 (Precision X-Ray) equipped with a beam conditioning filter (1.5 mm aluminum, 0.25 mm copper and 0.75 mm tin) at a focus-to-surface distance (FSD) of 70 cm. Sham irradiation was performed by placing the cultured neurons at the centre of the X-ray irradiator for 2.5 minutes (i.e. the duration of X-ray irradiation at 150mGy) without switching on the machine. Alpha-particle and X-ray irradiation were performed one hour after cell plating.

### Neurite Outgrowth and Cell Survival Assays

For ex vivo DRG explant culture, non-overlapping quadrant images were taken from each DRG explant, and total neurite length was measured using automated WIS-NeuroMath software (Weizmann Institute of Science). Data were obtained from at least 3 separate experiments with n=8 explants per dose. For dissociated DRG cultures, DRG cultures were fixed with 4% paraformaldehyde (PFA) after 17 hours of incubation, blocked with 0.5% bovine serum albumin/0.1% Triton X-100 (Sigma-Aldrich), incubated with anti-βIII-tubulin (1:800; Sigma-Aldrich) and then incubated with corresponding secondary antibodies conjugated with Alexa Fluor 488 (1:300; Molecular Probes). Neurite outgrowth assay was performed as described. Briefly, 30 non-overlapping images were taken at 10× magnifications using an epifluorescence microscope (Nikon Eclipse Ni-E) equipped with a motorized stage. Total neurite length and average longest neurite length were quantified using automated WIS-NeuroMath software (Weizmann Institute of Science). Data was obtained from 3 independent experiments repeated in duplicates. βIII-tubulin-positive DRG neurons with intact neurites adjacent to the neuronal cell bodies were counted as healthy neurons for neurite outgrowth assay.

Cell survival assay was performed using WST-1 reagents according to the manufacturer's instructions (ClonTech). Briefly, DRG neurons were plated onto a poly-D-lysine (100 μg/ml) and laminin (10 μg/ml) (Sigma-Aldrich)-coated 96-well plates at a cell density of 500 cells per well, and incubated in full NB medium. After LDIR or siRNA treatments, 10 μl of WST-1 reagent was added into each well and incubated for another 3.5 h. The absorbance at 460 nm was determined using a microplate reader (BioTek Powerwave XS MQX200R). Data were obtained from three independent experiments repeated in triplicate.

### Genomic DNA Extraction and Reduced Representation Bisulfite Sequencing (RRBS)

After 17 hours of incubation, α-particle-treated or X-ray-treated DRG neurons were trypsinzed and resuspended with the cell lysis buffer. Genomic DNA was isolated using PureLink Genomic DNA Mini Kit (Invitrogen). Genomic DNA was digested with MspI, and preceded to library preparation. After bisulfite conversion which converted the unmethylated cytosine nucleotides into uracil, while methylated cytosine nucleotides remained unmodified, the bisulfite converted DNA was amplified using PCR, and the amplified PCR fragments was sequenced using Illumia HiSeq 1500.

Bioinformatics analysis on RRBS was performed as previously described. Briefly, raw reads of RRBS were first aligned to the mouse reference genome (UCSC GRCm38/mm10) using Bismark (version 0.13) and bowtie2 (version 2.3.5) program with default parameters. The resultant BAM files were then imported to the R package methylKit (version 1.2.10) to calculate DNA methylation levels of promoter regions (5,000 bp upstream of the transcription start site) for each gene. Several studies suggested that differential methylation is strongly enriched at the promoter region (5,000 bp upstream of the transcription start site), and thus putative promoter sequence of the gene is likely to be found, and its expression is affected by DNA methylation at the promoter region.

### Total RNA Extraction and qPCR Analysis

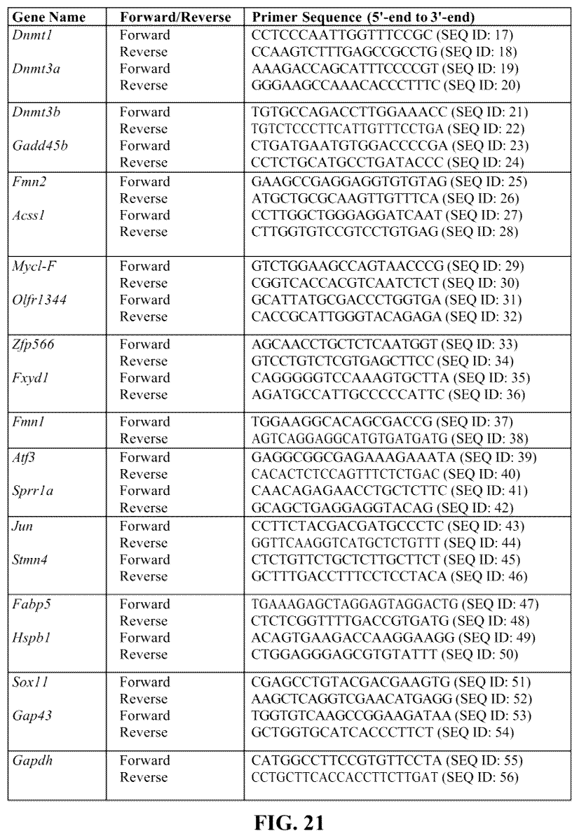

After 17 hours of incubation, total RNA was isolated from DRG cultures treated with 20mGy of α-particle irradiation or with 150 mGy of X-ray irradiation, and their respective sham irradiated controls, using Trizol reagent (Invitrogen). Lumbar 4 and 5 (L4/5) DRG neurons directly supplying the sciatic nerve injury were harvested at various time points after sciatic nerve transection, and total RNA was extracted using Trizol reagent (Invitrogen). Reverse transcription was performed using Superscript III First-Strand Synthesis SuperMix (Invitrogen). Quantitative PCR (qPCR) were performed in triplicates using KAPA SYBR Fast qPCR Kit (KAPA) on QuantStudio 12K Flex Real-Time PCR System (Applied Biosystems). Gapdh was used as an endogenous control, and the relative fold change of each gene candidates was calculated using 2^{-ΔΔct} formula. The qPCR primer sequences were listed in FIG. 21.

### AAV2/9-Mediated In Vivo Knockdown of Fmn2 and Hdac5 Expression

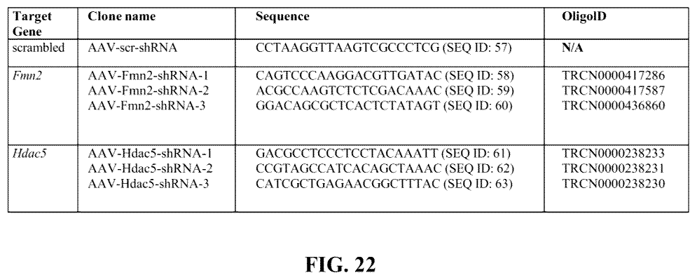

For in vivo gene silencing of Fmn2 and/or Hdac5 in DRGs, AAV2/9-Fmn2-shRNA and AAV2/9-Hdac5-shRNA at a titer of 5×10¹² vg/mL (OBIO Technology) were injected into the sciatic nerve directly through a glass micropipette using a microinjector (Harvard Apparatus) two weeks before sciatic nerve crush injury. The mice were then subjected to sciatic nerve crush injury and sciatic nerve pinch test (FIG. 4) and animal behavioural assessment (FIG. 15). The expression of shRNAs was under the control of the U6 promoter, and the expression of the reporter gene eGFP was under the control of the CAG promoter. AAV2/9-scr-shRNA (Packgene) was used as a negative control (see FIG. 22 for the shRNA sequences). On a separate batch of animals, the knockdown efficiency in DRGs was assessed by Western blot analysis in AAV2-Fmn2-shRNA and AAV2-Hdac5-shRNA mice two weeks after the AAV injection (FIGS. S8B and S9). To confirm the successful knockdown of Fmn2 in motor neurons, the lumbar ventral horns were harvested from in AAV2-Fmn2-shRNA mice for Western blot analysis (FIG. 14D) and immunohistochemistry study (FIG. 14E) two weeks after the AAV injection.

### Sciatic Nerve Pinch Test

Sciatic nerve pinch test was performed in the ipsilateral side 3 days after sciatic nerve crush injury. Mice were anesthetized under 2.5% isoflurane, and left sciatic nerve was exposed at the mid-thigh level. Mice were then slowly recovered under light anaesthesia (1% isoflurane). A series of pinches were applied from the most distal part of the sciatic nerve near to the trifurcation, moving proximally to the crushed site, using smooth forceps (Dumont #5/45 Forceps, Fine Science Tools). The distance (in mm) representing the length of axonal regrowth was recorded from the crushed site to the most distal point of sciatic nerve whereas initial withdrawal reflex was observed after pinching. The observers were blinded to the treatment and genotype.

### Quantification of In Vivo Axon Regeneration

Mice were perfused transcardially with 4% PFA, and L4/5 DRGs and sciatic nerves were harvested, post-fixed, cryoprotected and frozen in OCT compound (Tissue-Tek). For regenerating axon quantification, 12-μm thick longitudinal serial sections of sciatic nerve were blocked with 0.5% BSA/0.1% Triton X-100 (Sigma-Aldrich), and incubated with anti-SCG10 (a marker for regenerating sensory axons; 1:5,000; Novus Biologicals) primary antibodies. The sections were then incubated with secondary antibody conjugated with Alexa Fluor 555 (1:300; Molecular Probes). A series of z-stack images were taken at 20× magnifications using Nikon AXR confocal microscope equipped with a motorized stage and a resonant scanner, stitched, and maximally projected using NIS-Elements software.

To quantify regenerating axons, the SCG10 fluorescence intensity was measured at different distal distances away from the crush site using a line scan macro in ImageJ (NIH). A fluorescence intensity profile of SCG10 immunoreactivity was plotted by averaging the SCG10 intensity from 100 neighbouring pixels and normalized to that measured in the crushed site with maximal SCG10 immunoreactivity. For each mouse, at least 3 non-overlapping sections (at least 36 μm apart) were used and averaged from 12-15 sections per genotype or treatment group (n=4-5 per genotype or treatment group).

### Sensory and Motor Function Recovery Tests

Adult male Fmn2^{-/-} mice (8-12 weeks old) and their age-matched WT littermates, or adult male C57BL/6 mice (for whole-body X-ray, AAV2/9-Fmn2-shRNA and metaxalone experiments) were trained and habituated one week before the sciatic nerve crush injury. After three independent training sessions (30 minutes per session with two days break), baseline values were taken, and sciatic nerve crush injury were performed one day after the baseline recordings. Function recovery was monitored for 30 days. On day 3 after the sciatic nerve crush, we first perform pinprick assay and followed by motor function assays every other day in a sequential manner (toe spreading, grip strength, and sciatic functional index) with 30 mins apart from each test. Animal behavioural tests were done blinded to the surgery and genotypes.

### Pinprick Assay

Mice were placed singly in small wire-mesh cages for 30-minutes habituation, an insect pin (Fine Science Tools) were applied to the lateral plantar surface of the ipsilateral hindlimb from toe to heel. The lateral planar surface was divided into 5 different areas, from the most distal toe (score=5) to the heel (score=1). The mouse was scored for that area if a brisk withdrawal response of hind paw was observed. The mouse was scored 0 if no withdrawal responses were observed after insect pin was applied to all 5 areas. Saphenous innervated area of the same hindlimb was tested as a positive control if there was no positive response from these five areas. Two rounds of pinprick assay were performed (with a 30-minute time interval) in each mouse to confirm the score.

### Toe Spreading Reflex

To assess toe spreading reflex following PNI, mice were covered by a piece of cotton, lifted up by the tails, and uncovered their hindlimbs for assessment. Toe spreading reflex was scored as follow: 0 for no spreading, 1 for intermediate spreading with all toes separated for less than 2 seconds, and 2 for full spreading with all toes completely and widely spread, and sustained for at least two seconds. Mice were scored only when a full response was observed on the contralateral side (uninjured hindlimbs). Mice were assessed twice with 30-minute time interval in between.

### Grip Strength Measurement

Muscle strength from hindlimbs were measured using a grip strength meter (BIO-GS3, Bioseb). To measure the grip strength of both hindlimbs, their hindlimbs were positioned to grip a metal T-bar connected to the grip strength meter, while their forelimbs were rested on a plastic bar. The mice were then gently pulled off from the T-bar. The values at which the mouse left the T-bar reflected the grip strength in grams. The average grip strength was taken from at least 5 consecutive grip strength measurements per animal.

### Sciatic Functional Index (SFI)

The walking track analysis was performed by training the mice to walk down a narrow corridor covered with white paper strip (10×60 cm) and the hindlimbs of the mice were pained with red water colour. SFI baseline values were taken after 3 independent training sessions spanning across the week before injury. The SFI values were calculated from footprints as follows:
38.3×(experimental print length−naïve print length)/naïve print length+109.5×(experimental toe spread−naïve toe spread)/naïve toe spread+13.3×(experimental intermediary toe spread−naïve intermediary toe spread)/naïve intermediary toe spread−8.8. Print length was considered as the distance between the third toe to the heel. Toe spread was considered as the distance between the first and the fifth toe. Intermediary toe spread was considered as the distance between second and forth toe. Four clear and distinct footprints were taken from both the left injured ipsilateral experimental paw and the right uninjured contralateral naive paw for SFI calculation. Mice with SFI values close to 0 indicates normal gait movement, while the motor function is severely impaired if the SFI values close to −100.

### Electromyography (EMG) Recording

Electromyography (EMG) recording was performed on anesthetized mice as described. Briefly, mice were anesthetized with ketamine (100 mg/kg)/xylazine (10 mg/kg), and placed on a heat pad set to 37° C. to avoid hypothermia during the course of EMG recordings. Baseline EMG were recorded before sciatic nerve crush injury using custom-made monopolar Teflon-coated electrodes. Following sciatic nerve crush injury, EMG were recorded on a weekly basis for one month (days 7, 14, 21 and 28). For EMG recordings of proximal gastrocnemius muscle, recording electrode was inserted into the gastrocnemius muscle, and Achilles tendon electrode as a reference. For EMG recordings of distal interosseous muscle, recording electrode and reference electrode was inserted into first and fourth muscle of the same paw, respectively. Sciatic nerve was then stimulated proximally or distally, and both proximal and distal stimulations were used for CMAP of interosseous muscle. Only proximal stimulation was used for CMAP of the gastrocnemius muscle. The mean CMAP amplitude was recorded (Blackrock microsystem, USA) and calculated (Spike 2, UK).

### Immunohistochemistry Analysis of the Neuron Specific AAV Serotype Tropism in DRGs, Sciatic Nerves, and Lumbar Ventral Horn Motor Neurons

Two weeks after injection of AAV2/9-scr-shRNA (tagged with eGFP) into the sciatic nerves, mice were perfused transcardially with 4% PFA. Sciatic nerves, L4/5 DRGs, and lumbar ventral horn of spinal cords were harvested, post-fixed, cryoprotected, and frozen in OCT compound (Tissue-Tek). Five-micron-thick DRG cryosection, twelve-micron-thick longitudinal sciatic nerve cryosections, and thirty-micron-thick transverse spinal cord cryosections were blocked with 0.5% BSA/0.5% Triton X-100 (Sigma-Aldrich), and incubated with anti-βIII-tubulin (a neuronal marker; 1:800; Sigma-Aldrich), anti-ChAT (a motor neuron marker; 1:100; Millipore), anti-FMN2 (ProteinTech; 1:200), anti-SOX10 (a Schwann cell marker; 1:250; Abcam) or CD68 (a marker for macrophage; 1:250; Abcam) primary antibodies as indicated. The sections were then incubated with secondary antibodies conjugated with Alexa Fluor 555 (1:300; Molecular Probes) or Alexa Fluor 647 (1:300; Molecular Probes). For DRG and sciatic nerve cryosections, images were taken at 20× magnifications using Nikon Ni-E epifluorescence microscope equipped with a motorized stage (for DRG and sciatic nerve cryosections). For spinal cord cryosections, images were taken at 20× magnifications using Nikon AXR confocal microscope equipped with a motorized stage and resonant scanner.

### Fluorescence Activated Cell Sorting (FACS)

Two weeks after injection of AAV2/9-scr-shRNA or AAV2/9-Fmn2-shRNA into the sciatic nerves, L4/5 DRGs were harvested, digested with collagenase/dispase, mechanically dissociated in dissociation buffer (1× PBS with 10% FBS) using flame polished Pasteur pipettes with three different diameters, and then filtered through a 70-μm cell strainer (BD Falcon) to yield single cell suspension before FACS. All AAV2/9 used in the currently study were tagged with eGFP.

FACS was performed using SONY SH800Z Cell Sorter. To select DRG neurons, the dissociated cells were first separated based on the cell size (forward scatter) and cell surface characteristics (side scatter), and aggregated cells were eliminated based FSC-H:FSC-A ratio (FIG. 14A). In each experiment, dissociated DRG neurons without AAV transduction were used as negative controls to set up the detection gate for eGFP-positive cells (FIG. 14B). The sorted eGFP-positive cells were immediately lysed and subjected to Western blot analysis.

### Western Blot Analysis

Ipsilateral L4/5 DRG neurons were harvested at various time points following sciatic nerve injury as indicated. Sciatic nerves were harvested 3 days after crush injury and segmented into 0-6 mm distal to the crush site. Each protein sample was pooled from 3 mice. DRG neurons and segmented sciatic nerves were mechanically dissociated with protein lysis buffer, and subjected to measurement of protein concentration using protein BCA assay (Pierce). Protein lysate (30 μg) was separated on 4-12% NuPAGE Tris-Glycine precast gel (Invitrogen). Protein was then transferred onto a PVDF membrane (Bio-Rad) and blocked with 5% non-fat milk (Bio-Rad) in TBS-T. The membrane was incubated with anti-FMN2 (1:500; ProteinTech), anti-phosphorylated HDAC5 (1:500, ThermoFisher), anti-HDAC5 (1:500, Novus Biologicals), anti-acetylated tubulin (1:10,000; Sigma-Aldrich), anti-NeuN (1:500; Millipore), anti-SOX10 (1:500; Abcam), anti-IBA-1 (1:1,000; Wako), anti-GFP (1:1,000; Cell Signaling), and anti-GAPDH antibodies (1:2,000; ProteinTech) as loading controls. The membrane was incubated with secondary antibody conjugated with horseradish peroxidase (1:2,000; Thermo-Fisher). Signals were detected using West Femto Maximum Sensitivity Substrate Kit (Thermo Scientific). The membrane was then stripped and re-blotted with anti-total tubulin antibodies (1:2,000; Sigma-Aldrich). Band intensities were measured using Image Lab software (Bio-Rad). FMN2 and pHDAC5 protein expression levels were normalized to GAPDH. Acetylated tubulin protein expression levels were first normalized to total tubulin levels, and then normalized to GAPDH.

### Live-Cell Imaging and Analysis of Microtubule Dynamics at the Growth Cone of DRG Neurons

Two thousand DRG neurons were plated onto a poly-D-lysine and laminin-coated glass bottom dish, and transduced with AAV2/9-EB3-mCherry (Packgene) one hour after plating. On day 4 in vitro, EB3-mCherry tracking was performed and time-lapse images were taken at 100× magnifications using Nikon AXR confocal microscope equipped with a motorized stage and resonant scanner. Kymographs of EB3 comet motions at the growth cones were generated using NIS-Elements software. For microtubule dynamics analysis, the EB3 comets at the growth cones were automatically traced and parameters of microtubule dynamics, including microtubule growth velocity, growth lifetime, growth length, and EB3 comet density, were determined using a MATLAB-based software plusTipTracker. For each experimental group, at least 30 growth cones were analyzed from three independent experiments.

### HDAC5 Inhibitor LMK-235 Treatment

To inhibit HDAC5-mediated tubulin deacetylation in mice, a selective inhibitor of HDAC4 and HDAC5, LMK-235 (20 mg/kg), was intraperitoneally injected into the adult male C57BL/6 mice (8-12 weeks old) pre-treated with AAV2/9-Fmn2-shRNA for 3 consecutive days. The mice were then subjected to sciatic nerve pinch tests and immunohistochemistry analysis.

### RNA-Seq Whole Transcriptome Analysis

Sciatic nerve transection was performed in adult male Fmn2^{-/-} mice and WT littermates (8-12 weeks old), and L4/5 DRGs were harvested from both contralateral (uninjured) and ipsilateral (injured) sides 5 day after injury. Total RNA was extracted using Trizol reagent, and 100ng of total RNA from each sample (n=3 per group) was used for library preparation after ribosomal RNA depletion. The library of each sample was then sequenced using Illumina Novaseq 6000 to a depth of 75 million reads per sample. After quality control using FastQC, sequencing data was mapped and annotated with the reference genome GRCm38, and quantified using DESeq. Genes with log2-fold>|1| and Benjamini-Hochberg adjusted P<0.05 were considered as differentially expressed. DESeq2 normalized counts with log2 transformation were used for principal component analysis and hierarchical clustering analysis.

### Functional Enrichment Analysis

To examine the biological functions associated with differentially expressed genes in Fmn2^{-/-} mice, GSEA and gene set overrepresentation analysis were performed on Gene Ontology (GO) gene sets (biological process, cellular component and molecular function categories) and KEGG pathways using ‘HTSanalyzeR’ R package. Significantly enriched GO terms and pathways (Benjamini-Hochberg adjusted P-value<0.05) were clustered by AutoAnnotate (Version 1.3.2) with Markov Cluster method and visualized using Cytoscape (v.3.7.2).

### In Silico Small Molecule Screening

Library of Integrated Network-Based Cellular Signatures (LINCS) database was used for in silico small molecule screening. LINCS is a public web-based database which contains more than 1 million gene expression profiles derived from 50 human cell lines (including neuronal cell lines such as fibroblast-derived neuronal progenitor cells and terminally differentiated neurons) after treating with 44,328 bioactive small molecules (including 1,234 FDA-approved small molecules). A total of 145 genes were uniquely differentially expressed only in injured Fmn2^{-/-} DRG neurons and were used as query signature, compared with the gene expression profiles of neuronal cell lines in the public LINCS database. Based on a non-parametric, rank-based pattern-matching Kolmogorov-Smirnov statistics, each of the query signatures in the gene expression profile of injured Fmn2^{-/-} DRG neurons was estimated with a metric, and the similarity of the query signature was evaluated with the gene expression profile derived from cell lines treated with small molecules (reference signatures). This generated a connectivity score ranging from +100 (strong positive correlation) to −100 (strong negative correlation). The mean of the connectivity scores was used to rank the small molecules. Small molecules with connectivity score greater than +95 (strong overlapping between the query signatures and the reference signatures) were shortlisted for experimental validation using sciatic nerve pinch test in mice.

### Assessment of Small Molecules for In Vivo Axon Regeneration and Function Recovery after PNI

To assess the promoting effects of top-ranked small molecules on in vivo axon regeneration, 40 μl of alverine (0.5 μg/μl), cilnidipine (0.5 μg/μl), metaxalone (0.25 μg/μl) or acamprosate (0.25 μg/μl) was administrated directly to the site of injury of adult male C57BL/6 mice (8-12 weeks old) immediately after the sciatic nerve crush for 3 consecutive days (with both morning and evening dosing regimens over an 8-hour time interval per day), given that the half-life of small molecules ranged from 5.7 to 33 hours (i.e., metaxalone 9 hours). The mice were then subjected to sciatic nerve pinch test and immunohistochemistry analysis.

To examine the therapeutic potential of metaxalone, adult mice received daily intraperitoneal injection of metaxalone at 10 mg/kg immediately after injury for 21 consecutive days (i.e., metaxalone treatment) or delaying daily intraperitoneal injection of metaxalone at 10 mg/kg to 24 hours post-injury for 20 consecutive days (i.e., delayed metaxalone treatment), and all metaxalone treatments ended on day 21 post-injury. The mice were then subjected to sensory and motor function recovery tests and weekly EMG recordings for 28 days post-injury.

### Statistical Analysis

Data are presented as mean±SEM. Student's t-test (2 groups) or one-way ANOVA (more than 2 groups) followed by post hoc Bonferroni tests was used for data analysis where appropriate. Two-way ANONA, followed by post hoc Bonferroni tests was used for the analysis of SCG10 immunoreactivity. Non-parametric Mann-Whitney U test (2 groups) or Kruskal-Wallis test followed by Dunn's post hoc tests (more than 2 groups) was used for the analysis of qPCR experiments. Two-way ANOVA with repeated measures, followed by post hoc Bonferroni tests was used for analysis of animal neurobehavioral and electrophysiological experiments. The microtubule growth velocity, microtubule growth length, microtubule growth lifetime, and the EB3 comet density were analyzed using Mann-Whitney U test. P<0.05 was considered to be statistically significant. GraphPad Prism 9.0 was used for all the statistical analyses and graphing.

### Data and Code Availability

Sequencing data are deposited in Gene Expression Omnibus (GEO) under the accession number GSE243896 and publicly available as of the date of publication.

What is claimed is:

1. A method of treating a nerve injury in a subject in need thereof, the method comprising: administering a therapeutically effective amount of a therapeutic agent to the subject, wherein the therapeutic agent is selected from the group consisting of metaxalone and a Formin-2 (Fmn2) inhibitor.

2. The method of claim 1, wherein the Fmn2 inhibitor is an antibody, an antibody fragment, an inhibitory nucleic acid molecule, or a small molecule, wherein the inhibitory nucleic acid molecule substantially silences Fmn2.

3. The method of claim 2, wherein the inhibitory nucleic acid molecule is selected from the group consisting of short interfering nucleic acid (siNA), a short interfering RNA (siRNA), a double-stranded RNA (dsRNA), a micro-RNA (miRNA), a short hairpin RNA (shRNA), a short interfering oligonucleotide, a short interfering nucleic acid, and a post-transcriptional gene silencing RNA (ptgsRNA).

4. The method of claim 2, wherein the inhibitory nucleic acid molecule comprises contiguous nucleotides complementary to a nucleic acid sequence selected from the group consisting of SEQ ID NO:1, SEQ ID NO:2, SEQ ID NO:3, SEQ ID NO:4, SEQ ID NO:5, SEQ ID NO: 6, SEQ ID NO:7, SEQ ID NO:8, and SEQ ID NO:9.

5. The method of claim 2, wherein the inhibitory nucleic acid molecule comprises SEQ ID NO: 10, SEQ ID NO:11, or SEQ ID NO:12.

6. The method of claim 2, wherein administration of the inhibitory nucleic acid molecule reduces Fmn2 protein expression by 40-100%.

7. The method of claim 1, wherein the therapeutic agent is administered within 24 hours of onset of the nerve injury.

8. The method of claim 1, wherein the therapeutic agent is administered parenterally, paracancerally, transmucosally, transdermally, intramuscularly, intravenously, intradermally, subcutaneously, intraperitonealy, intraventricularly, intracranially, perineurally, intraneurally, or directly at a central nervous system lesion site or a peripheral nervous system lesion site comprising the nerve injury.

9. The method of claim 3, wherein the inhibitory nucleic acid molecule is administered directly at a central nervous system lesion site or a peripheral nervous system lesion site comprising the nerve injury.

10. The method of claim 1, wherein the therapeutic agent is metaxalone.

11. The method of claim 1, wherein the nerve injury comprises at least one of a central nervous system injury or a peripheral nervous system injury.

12. The method of claim 1, wherein the nerve injury is the result of physical trauma.

13. The method of claim 1, wherein the nerve injury comprises an injured dorsal root ganglion.

14. The method of claim 1, wherein the subject is a human, a non-human primate, an equine, a bovine, a canine, a feline, or a rodent.

15. The method of claim 10, wherein the subject does not suffer from a muscle related condition.

16. The method of claim 15, wherein the muscle related condition is selected from muscle spasms, muscle spasticity, and musculoskeletal pain.

17. The method of claim 10, wherein the nerve injury is the result of physical trauma.

18. The method of claim 10, wherein administration of metaxalone begins within 24 hours of onset of the nerve injury.

19. The method of claim 10, wherein metaxalone is administered orally.

20. The method of claim 10, wherein the subject is a human.
